(19) United States
(12) Patent Application Publication
(10) Pub. No.: US 2021/0179467 A1
(43) Pub. Date: Jun. 17, 2021

(54) Sustainable processes for treating wastewater

(71) Applicant: Steven L Cort, Cary, NC (US)

(72) Inventor: Steven L Cort, Cary, NC (US)

(21) Appl. No.: 16/974,096

(22) Filed: Sep. 30, 2020

Related U.S. Application Data
(63) Continuation-in-part of application No. 15/732,401, filed on Nov. 7, 2017, now abandoned.

(51) Int. Cl. C02F 9/00; C05F 7/00; C12N 1/20

(52) U.S. Cl. CPC C02F 9/00; C02F 1/56; C12N 1/20; C05F 7/005

(57) ABSTRACT

The present invention relates to a new and novel process that combines treatment methods that use magnetite, Hydrothermal Carbonization (HTC), Hydrodynamic Cavitation (HDC), probiotics, and adsorption using Magnetic Hydrochar (MHC) and Water Treatment Residuals (WTR) to replace Activated Sludge Technology (AST) for the treatment of wastewater containing dissolved organic and inorganic contaminants.

### CROSS REFERENCE TO RELATED APPLICATIONS

This application is a continuation-in-part of Ser. No. 15/732,401, filed Nov. 7, 2017, and also claims priority from provisional application 63/102,956, filed Jul. 13, 2020.

### FIELD OF THE INVENTION

The present invention relates to sustainable processes for treating wastewater with the goal being to replace conventional Activated Sludge Technology (AST) with a combination of processes. In one aspect of the invention, the wastewater conveyance system is converted into a wastewater treatment device by delivering adsorbents and microorganisms such as: (1) Magnetic Hydrochar (MHC) produced using Hydrothermal Carbonization (HTC) to treat magnetic floc from a Magnetic High Rate Clarification (MHRC) system, (2) Mixed Liquor Suspended Solids (MLSS) from a Wastewater Treatment Plant (WWTP), (3) probiotics grown using “process water” from HTC or other waste biosolids generated by AST or MHRC, (4) probiotics produced from magnetic floc that has been disinfected and lysed by Hydrodynamic Cavitation (HC), and (5) Water Treatment Residuals (WTR) from iron coagulants used to clean water. Preferably these materials are delivered through a pipeline installed inside the wastewater conveyance system. The treated wastewater is then clarified with a MHRC system.

### BACKGROUND OF THE INVENTION

There are many known processes for treating wastewater and waste sludges. The most sustainable processes are those that combine technology with nature. A sustainable solution for a developed country may be highly technical; for example, the main focus may be to remove nutrients such as phosphorus and nitrogen to prevent eutrophication of waterways. However, this approach may not be sustainable for developing countries because of such factors as culture, economics, and infrastructure, and therefore developing countries have to depend on holistic solutions that conserve fresh water resources and recover resources to produce food.

Typical contaminants in wastewater are either Total Suspended Solids (TSS) or Dissolved Contaminants (DC). The predominant method for removing TSS from municipal wastewater is gravity clarification, but this method has significant flaws such as large footprint, low efficiency of solids removal in primary clarifiers, and problems with floating floc and flow restrictions in secondary clarifiers. A preferred approach is to use flocculating polymers in combination with weighted ballast material such as sand or magnetite. In ballast treatment applications, TSS is attached to the ballast material with the use of flocculating polymer. This floc is then mechanically dispersed, separating the ballast and TSS, so the ballast can be recovered and reused and the TSS disposed of.

Using magnetite as a ballast in place of sand has two significant advantages. First, magnetite ballast can be easily removed from wastewater magnetically, which produces a more concentrated waste (ten times more concentrated than sand ballast systems). Second, a magnetite system is smaller in physical size because magnetic floc is heavier than sand floc and will settle more rapidly in an existing clarifier. Alternatively, the magnetic floc can be removed magnetically, which is faster than gravity settling. The process of clarifying wastewater using magnetite in this patent application is called Magnetic High Rate Clarification (MHRC).

One drawback with using ballast material to speed clarification is that the ballast material, whether sand or magnetite, may become fouled with organics such as oil and grease. For ballast material to be suitable in applications that could cause fouling of the ballast material such as in the treatment of sanitary wastewater that can contain oil and grease, it is important that the method for regenerating the ballast material produces a clean ballast that can be reused. This is one reason why ballast clarification is not commonly used in treating raw sewage that contains fats, oils, and greases (FOG).

The state of the art for cleaning ballast material is a vortex cyclone for sand ballast or mechanical shearing for magnetite, which breaks the bond between ballast and TSS formed by flocculating polymer. Mechanical processes are not completely effective in cleaning ballast material that has been fouled with FOG or biofilm.

According to the present invention, a new method for regenerating ballast material used to clarify water is provided, and is referred to herein as Hydrothermal Carbonization (HTC). HTC uses heat and pressure to carbonize wet organic wastes to produce a useful product referred to in the industry as HydroChar (HC). When the organic waste is magnetic because it contains some form of magnetite or other magnetic material, a Magnetic HydroChar (MHC) product is produced.

One commercial advantage of the present invention is that the combination of MHC produced from magnetic floc originating from MHRC to adsorb DC and followed by MHRC to clarify wastewater, preferably while in its conveyance system, is an economic replacement for Activated Sludge Technology (AST) for treating organic-laden wastewater.

Heretofore, AST has been the technology of choice for treating wastewater containing organics, because it is usually the lowest cost option for biologically removing pathogens and dissolved nutrients from wastewater before being discharged into the environment and because it has a smaller footprint than lagoon treatment.

FIG. 7a shows a state of the art wastewater treatment plant (WWTP) employing AST. Wastewater (90) flows into a primary clarifier (91) where TSS are allowed to settle out by gravity in the form of sludge, which is periodically removed and is either discharged as Waste Activated Solids WAS (96) or used in an anaerobic digester (not shown) to produce methane for electricity generation. Clarified water from the Primary Clarifier (91) is then biologically treated in an aerobic Biological Treatment System (92) where bacteria convert DC into biosolids called Mixed Liquor Suspended Solids (MLSS). The MLSS then flows to a Secondary Clarifier (93) where solids are separated and used as Returned Activated Solids (RAS) (95) for Aerobic Biological Treatment (92) or Waste Activated Solids (WAS) (96) for Dewatering (97) and Disposal (98). Clarified water from the Secondary Clarifier (93) is then disinfected by UV (94) or another method and discharged (100). Primary and secondary solids are dewatered (97) and filtrate (99) is returned to the head of the AST for additional treatment and Biosolids (98) are normally disposed of.

However, AST is not without serious economic and environmental flaws. AST is known to produce large volumes of biosolids that are usually either disposed of in landfills or land applied. Either of these disposal practices result in high costs and serious environmental problems. Also, AST, because it is an aerobic process, requires large quantities of oxygen in the Aerobic Biological Treatment step (92), resulting in high electrical usage. Additional disadvantages of AST are: (1) large physical footprint compared to the process of this invention, (2) large carbon footprint, (3) excessive emissions of greenhouse gases and odors, and (4) complicated operation.

Furthermore, biological processes such as AST can only treat contaminants that are biodegradable. Since Contaminants of Emerging Concern (CEC) (i.e. toxic organics, pesticides, hormones, biocides, heavy metals, and pharmaceuticals) are not easily biodegraded and their toxicity can adversely affect the biological process, it is known in the art that AST is not especially effective in treating CEC contained in wastewater.

Adsorption using MHC or HC according to the present invention is not a biological process and therefore is effective in removing CEC. In practice, MHC or HC can be tailored to target and remove certain CEC, heavy metals, and even certain nutrients such as phosphorus or nitrogen

One goal of this invention is the redirection of carbon from a waste to a resource, which will improve the economics of wastewater treatment. With AST, energy is used to aerate bacteria and in this process greenhouse gases such as carbon dioxide, methane, and nitrous oxide are emitted. Furthermore, the burning of fossil fuel to produce electricity to aerate wastewater causes further emission of greenhouse gases, contributing significantly to global warming. Many larger Wastewater Treatment Plants (WWTPs) are equipped with anaerobic digesters to convert Mixed Liquor Suspended Solids (MLSS) into methane, which is combusted to produce electricity. In this process, carbon dioxide and fugitive methane emissions are lost to the environment. Therefore, significant amounts of energy are expended in practicing AST and significant greenhouse gases are emitted. A preferred energy efficient approach is to replace AST with MHC according to the invention to remove DC contained in wastewater, preferably as the wastewater flows through a wastewater conveyance system, and then to remove all solid contaminants in a MHRC. This reduces all the disadvantages of AST.

For example, AST requires a large physical footprint because bacteria are slow acting and MLSS produced by AST are lightweight and do not settle rapidly by gravity, thus requiring large clarifier tanks. By comparison, attaching magnetite to solids in the wastewater by use of a flocculating polymer has proven to be effective in increasing the speed of gravity separation because the specific gravity of magnetite is over five times the specific gravity of water. Sand ballast is also effective to clarify water but sand has only half the specific gravity of magnetite, requiring more space, and, as mentioned above, the sand ballast cleaning process using hydrocyclones produces a large volume of dilute waste, typically ten times more than produced in processes using magnetite.

The concentration of TSS in raw sewage entering AST is typically about 300 ppm. The concentration of TSS in the form of MLSS exiting AST treatment is about 3000 ppm. This great increase in TSS is attributed to the growth of bacteria biomass as bacteria consume DC contained in sewage. Because the treatment processes to disinfect MLSS biosolids, and the processes to break down toxic organics and remove heavy metals contained in MLSS, are not completely effective, land application of MLSS is not without significant environmental problems and opposition. Replacing AST with MHRC, HTC, probiotics, Water Treatment Residuals (WTR) and MHC according to the methods of this invention would substantially eliminate most concerns over waste disposal and its impact on the environment.

Each year, over 20 million tons of MLSS biosolids are produced globally and this does not include the amount of untreated sanitary wastes presently discharged into the environment or the amount of sanitary wastes collected in septic tanks (septage). Reducing the volume of waste solids and therefore the cost of treating these organic wastes by recovering valuable resources will decrease the amount of waste that is allowed to contaminate the environment. Recovering phosphorus is especially important because it is necessary for plant growth and known mineable reserves are dwindling. Therefore, replacing AST, which is not cost-effective and causes significant environmental problems, with more efficient technologies such MHRC (clarification) and HTC to produce MHC to adsorb DC as described in this application offers significant economic, health, and environmental benefits, especially where it is sorely needed in developing countries.

FIG. 7b shows a schematic diagram of an AST replacement system according to this invention that reduces cost and operating complexity and is especially suitable for applications in developing countries. The individual components of the system are detailed below. In brief, wastewater (101) flows into a MHRC (102) where TSS form a magnetic floc using a flocculating polymer, and then magnetic floc flows to a HDC (103) where the TSS are disinfected and lysed, producing Hydrochar (HC). Flow from the HDC (103) goes to a Probiotic Production system (105) that grows probiotics using the lysed organics. Water from the MHRC is sterilized by UV or the like at (104). Probiotics and magnetite are pumped upstream into the Influent Conveyance System (107) and probiotics only are pumped into the effluent line (106).

The advantages of the present invention will now be discussed, followed by a detailed explanation of the components of the system.

Hydrochar (HC), the solid material produced from the HTC process, is conventionally produced using wet organic wastes such as WWTP biosolids, food wastes, septage, and grease trap wastes, and can be used as a low quality fuel such as lignite or wood chips. This approach is proven (Suez and Terra Nova Slovenia project) (see YouTube—Hydrothermal Carbonization with Terrallova Ultra as Innovative Sewage Sludge Treatment), and can certainly provide a beneficial use for the vast quantities of biosolids produced by a WWTP, but the economic return is low. Low quality solid fuels such as lignite or wood chips are worth between $30-$70 per ton and involve significant added transportation expenses, while their burning causes emission of carbon dioxide. Without carbon credits, producing a low value fuel from WWTP biosolids is not likely to be economical.

Another HC use is land application as a soil amendment to increase food production and to sequester carbon, but this alternative also has low economic return and there are also some air emission concerns about applying HC to the land.

A more economical use for HC biosolids it to use them as an adsorbent for cleaning water or air. With additional chemical or mechanical processing, HC can be converted into activated carbon, which is worth between $700-$1500 per ton. HC is highly carbon-concentrated and therefore transportation costs are kept to a minimum.

An improved form of HC is Magnetic Hydrochar (MHC), which is useful in removing contaminants from water, and which has magnetic properties that allows it to be easily removed from water magnetically and regenerated. The addition of iron or other chemical catalysts to MHC also improves its adsorbency to remove heavy metals and dissolved organics.

It is known in the art that magnetite is proven to be an effective ballast material to improve clarification of wastewater because magnetite will bond to TSS with the use of a flocculating polymer to form a magnetic floc. One known practice, as demonstrated by the Comag technology offered by Evoqua Water Technologies LLC and described in their website (Evoqua.com—The Comag System for Enhanced Primary and Tertiary Treatment) to recover the magnetite ballast is to break floc bonds by mechanical means so the magnetite can be reused and the TSS disposed of as a waste. The use of magnetite has been shown to be useful in removing phosphorus from sewage and can double the capacity of a WWTP without increasing the size of its footprint.

It is also known that Suez and Terra Nova completed a commercial project that used HTC to convert WWTP MLSS, a high moisture waste, into HC, which is used as an alternative fossil fuel. However, it has not been contemplated to retrofit the use of magnetite into the WWTP to produce a magnetic floc that contains organics and magnetite to be used in HTC to produce MHC for treating water according to the present invention, instead of producing MLSS at the WWTP to produce HC to burn as an alternative fuel. The alternative process described herein is advantageous because it: (1) increases the capacity of the WWTP, (2) reduces the emissions of greenhouse gases and odor from the WWTP, (3) eliminates the need to mechanically separate the magnetic floc from the treated wastewater so the magnetite can be reused, (4) produces a more valuable byproduct (MHC is more valuable than low quality solid fuel), (5) reduces energy usage, and (6) reduces operating problems (odor, corrosion, and FOG buildup) in the wastewater conveyance system if MHC is injected upstream in the wastewater conveyance system. In summary, according to this invention, instead of producing an organic waste (MLSS biosolids) for disposal in a landfill or producing a low value solid fuel, a novel process is provided allowing conversion of the WWTP into a system that uses magnetite to produce magnetic floc that is then converted into MHC, a valuable water treatment product that when injected into the wastewater conveyance system will reduce operational problems such as odor, corrosion, and FOG buildup.

HTC uses low temperature (150-250 degrees F.) and low pressure (1-2 bar) to convert wet carbon wastes into HydroChar (HC), a valuable adsorbent. According to this invention, when magnetic floc from MHRC is carbonized in HTC, MHC, a solid carbon product that has excellent adsorption properties for removal of DC from wastewater is produced, while the HTC “process water” (water resulting from HTC) is high in nitrogen, phosphorus, and potassium, all the nutrients necessary for plant growth.

Likewise, HTC “process water” contains all the essential nutrients needed to grow probiotics that can be used to biologically treat biodegradable DC in wastewater and which when applied to the wastewater conveyance system will reduce odor, corrosion, and the buildup of fats, oils, and greases (FOG). Therefore, HTC produces two byproducts (MHC and “process water”) that are used to reduce problems in wastewater conveyance systems and in particular as a pretreatment to MHRC that produces a process that effectively replaces AST.

As mentioned, a common reuse option for biosolids (MLSS) produced in conventional AST processes is land application, and while biosolids are an important source of nutrients for plant growth, using biosolids is not without environmental problems or opposition. Biosolids from AST may contain: (1) high levels of heavy metals, which over time when land applied can exceed the natural capacity of the soil to support healthy plant growth, (2) excessive amounts of phosphorus that can cause waterway eutrophication from runoff, (3) nitrates that can potentially contaminate ground water, (4) high greenhouse gas and odor emissions, and (5) high levels of recalcitrant toxic organics. Treatment methods presently used for biosolids such as composting or lime stabilization do not degrade toxic chemicals, do not remove microplastics, and do not completely disinfect biosolids. Therefore, a superior method for the treatment of MLSS biosolids is HTC, which removes all traces of toxic organics, microplastics, and pathogens.

This patent application also presents the use of HDC to treat organic solid waste but specifically magnetic floc from MHRC to: (1) make it possible to reduce heavy metal concentrations, (2) recover phosphorus, (3) provide a safe nutrient-rich organic fertilizer for food production, (4) reduce the particle size of magnetite to make it a more effective adsorbent, and (5) disinfect and lyse waste carbon and WWTP MLSS, where available, to provide a source of food to grow probiotics.

In summary, due to global warming concerns resulting from greenhouse gas emissions, the poor energy efficiency of biological treatment of organic contaminants in wastewater, and the large amount of presumably toxic biosolids generated, AST is no longer the best way to treat organic wastewater economically and in an environmentally-friendly way, especially in developing countries. According to this invention, a new process is provided that uses MHRC to remove TSS from wastewater, preferably while it is flowing through its conveyance system, and then uses HTC to convert magnetic floc from MHRC into MHC. This method is preferable to AST for removal of DC from wastewater since it: (1) treats water faster and more economically, (2) uses less energy, (3) produces valuable byproducts that can clean water, (4) produces no waste, (5) has a smaller carbon footprint, (6) removes CEC, and (7) emits less greenhouse gas. The invention also provides a less capital-intensive alternative to AST by treating wastewater while in its conveyance system, again by clarifying the wastewater with MHRC to remove solids in the form of magnetic floc that is then treated with HDC so the magnetite can be reused, while probiotics can be grown on the removed biosolids and also used for in-line treatment in the wastewater conveyance system.

The use of MHC as an adsorbent to remove DC from wastewater takes time and adequate mixing. This can be accomplished in a tank but a better approach is to use the wastewater conveyance system itself as a tank and letting the MHC adsorb DC while the wastewater is flowing to the WWTP. However, this requires an effective delivery system to deliver the MHC as far upstream as possible to increase the residence time within the conveyance system to adsorb DC.

Certain distinct elements of the system of this invention are known individually in the art, such as the use of inline bioaugmentation and high-rate clarification. However, these known elements have never been combined with other elements provided according to the invention, such as increasing the holding capacity of the conveyance system by the use of flow control devices or growing select microorganisms at a WWTP and delivering the select microorganisms to the head of the conveyance system, to produce a unique combination of treatment elements to effectively biologically treat wastewater. By proper combination of these elements, this invention provides a system that can cost-effectively biologically treat wastewater.

More specifically, Dickerson in U.S. Pat. No. 5,788,814 discloses a bioaugmentation method for treating wastewater in its conveyance system prior to a WWTP. The Dickerson patent was the basis for starting a company named In-Pipe Technology, Inc. In-Pipe Technology uses the continuous addition of an extremely high concentration of microbes into the collection system to eliminate sewer system grease and odor problems, reduce sludge production, reduce electrical power requirements at the WWTP and to reduce infrastructure corrosion.

Dickerson did not contemplate growing the bacteria at a WWTP using biosolids produced at the WWTP, nor contemplate the delivery of bacteria from the WWTP via a pipeline contained inside the conveyance system, nor contemplate provision of flow control devices to hold up the flow of wastewater in the conveyance system to increase the residence time for biological treatment.

Dickerson in U.S. Pat. No. 5,788,814 discloses a bioaugmentation method for treating wastewater in its conveyance system prior to a WWTP, using dosing systems placed far upstream in the wastewater conveyance system. While effective, this approach is labor-intensive and resulted in operational problems with keeping the dosing systems maintained and supplied with probiotics. A better approach is to install a pipeline, preferably inside the wastewater conveyance system itself, that will transport MHC from a probiotic production system located at the WWTP to the head of the wastewater conveyance system at strategic locations. This same pipeline delivery system can be used to deliver WTR, probiotics grown on HTC “process water”, probiotics grown on magnetic floc using HDC, and MLSS biosolids from AST upstream near to the head of the wastewater conveyance system.

Williams et.al. U.S. Pat. No. 8,828,229 discloses a method of treating wastewater using a membrane biological reactor (MBR) to reduce the hydraulic load on a WWTP by providing a concentrated organic waste stream to the WWTP. The Williams patent describes the function of the MBR as an intermediate WWTP that biologically treats the wastewater in the conveyance system upstream from the WWTP so that clean water can be discharged and the concentrated biosolids flow to the WWTP for additional treatment. The Williams patent describes the benefit of this process for the treatment of municipal sanitary wastewater and does not apply to the treatment of inline biological treatment of wastewater. As noted above, the quantity of municipal sanitary wastewater increases during rain events, during which the treatment method expressed in the Williams patent would reduce the hydraulic load on the WWTP, but this is only accomplished by the use of membrane bioreactors positioned along the conveyance system. Williams' patent neither contemplates increasing the holding capacity of the conveyance system nor does it use high-rate clarification followed by disinfection if necessary for direct discharge of treated wastewater without the need for a WWTP.

Tests by SCD Probiotics in Cartagena, Colombia, South America proved that the use of probiotics in a conveyance system increased the efficiency of sewage treatment by extending the treatment time. In a sewage system 21 Km in length and having a typical residence time of 2.5 hours, TSS was reduced 68% and BOD reduced 66%

Another test by SCD Probiotics in Lowicz, Poland using probiotics showed a reduction of BOD from 623 ppm to 13.2 ppm with an overall 25% reduction in sludge production when sewage was treated inline.

The preferred bacteria contained in the mixture of microorganisms used in this invention are in the family of facultative bacillus that are non-pathogenic and non-toxic, which includes but is not limited to B. Subtiles, B. Licheniformes, B. Indicus, B. Coagulans, B. Cereus, and B. Clausii. These bacteria are ubiquitous in nature, especially in soil. They are facultative by nature, meaning that they do not need oxygen to survive and to be pioduelive. Therefore, they are effective in both aerobic and anaerobic environments and reduce odor and corrosion in the conveyance systems by consuming chemicals contained in the wastewater. If the mixture of facultative microorganisms flows to a WWTP, the need for aeration is reduced or completely eliminated and electrical usage can be reduced by as much as 50% because most WWTPs use aerobic bacteria that need high levels of oxygen and therefore incur high electrical costs for aeration.

Phages are viruses that infect and kill microorganisms. Sulfur reducing bacteria (SRB) cause the most harm in a water conveyance system because they convert sulfates contained in the water into sulfides that react to form hydrogen sulfide, a major cause of odor, and sulfuric acid, a major cause of corrosion. Phages infect and control the growth of SRB but can be selected to do so while not harming beneficial microorganisms.

The bioaugmentation performed in the conveyance system requires microorganisms that are either grown onsite or offsite. The preferred embodiment of this invention is to produce microorganisms at a WWTP, using biosolids in wastewater as food for the microorganisms.

A second aspect of the treatment process according to the invention is control of the flow of the wastewater to be treated to increase the holding capacity of the wastewater conveyance system, which will enhance the performance of inline bioaugmentation as discussed above. That is, during both dry and wet weather conditions, the flow control device is operated so as to control the residence time of wastewater in the conveyance system to provide sufficient time for the microorganisms to perform their function.

Flow control can take many forms but the preferred method is to provide a damming device containing an adjustable weir to control the residence time of the select microorganisms in the conveyance system, which can be an existing device converted according to the invention into a treatment system. This gives the microorganisms adequate time to act to reduce nutrient levels. The importance of flow control in a conveyance system results from the fact that the flow during wet weather events can be 10 to 100 times the flow in the conveyance system during dry weather. Without delaying some of the storm flow using a variable-height weir, the storm flow will be too great to allow the microorganisms enough time to biologically treat the wastewater. Another important function of the flow control device is to level out the flow of wastewater over a period of time so that the wastewater can be clarified and disinfected at a slower rate over a longer period of time. It is also within the scope of the invention, in order to increase the residence time for biological treatment and to save space, to extend the wastewater conveyance system along the edge of a riverbed like a canal. In some cases, the wastewater conveyance system can actually be positioned in the riverbed.

The third aspect of treatment according to the invention is high-rate clarification followed by disinfection if necessary. Clarification is necessary to meet total suspended solids (TSS) limits and since the TSS levels influence basic oxygen demand (BOD), which is a measure of the amount of organics present, and bacteria levels, fine suspended solids must be removed. The most effective method for fine solids removal is high-rate clarification using flocculating polymers. Because of the high flow rates during storm events, only high-rate clarification is applicable, primarily because of space and cost limitations. This invention contemplates the use of high-rate clarification methods that have a surface overflow rate (SOR) greater than 10 gallons per minute per square foot of surface area of the clarifier. For example, for a flow rate of 10 million gallons per day or about 7000 gallons per minute, the surface area of a high-rate clarifier with a SOR of 10 gallons per minute per square foot would need to be about 700 square feet. By comparison, if a traditional gravity clarifier with a SOR of one gallon per minute per square foot were used the clarifier would need to have a surface area of about 7000 square feet. Therefore, a significant saving in the space required is realized. The methods of high-rate clarification methods preferred for practice of this invention are dissolved air flotation, lamella gravity clarification, and ballast clarification using sand or magnetite, all of which are generally well understood in the art.

High-rate clarification produces a concentrated slurry of suspended solids in the approximate range of 1-5 weight % solids. This slurry can be dewatered by mechanical means to produce a dry cake that can be either landfilled or land applied. Another economical solution is to flow the concentrated slurry into a dewatering containment that will allow adequate time for the solids to settle and then be removed periodically as a more concentrated wet solid. This wet solid can be land applied but not landfilled unless it passes a paint filter drip test. Supernatant from the dewatering containment can be directly discharged assuming the total suspended solids are low enough to meet regulatory limits. However, the best solutions are to apply the concentrated slurry from the high-rate clarification system or the dewatering containment directly to the land to improve soil conditions or to a WWTP to increase biological treatment capacity assuming that regulatory limits are not exceeded.

Because of the sheer magnitude of the problem and the cost of adequate solutions, regulations regarding wastewater are constantly changing. It appears, according to present EPA regulations, that primary clarification and disinfection are acceptable solutions to the problem if bacteria levels can be sufficiently low and the BOD of the wastewater can be reduced by 85%. This may not be possible unless bioaugmentation in the conveyance system and containment structures is followed by flow control to increase the capacity of the conveyance system to achieve the necessary level of biological treatment, in turn followed by high-rate clarification using flocculating polymers according to this invention.

The combination of the treatment methods contained in this invention is arranged in such a way to meet regulation-specified discharge limits, which primarily specify acceptable levels of TSS, BOD, bacteria, and floatables. The level of BOD and bacteria are reduced significantly by the inline bioaugmentation within the conveyance system but if the bacterial levels are still too high, further disinfection may be performed as necessary, using methods including but not limited to employment of known disinfectants such as chlorine, peracetic acid, UV, and ozone.

WWTPs grow bacteria that are beneficial in the treatment of municipal wastewater. These bacteria use the organic wastes and other chemicals contained in municipal wastewater (CSO) as a food source, thus consuming the wastes and purifying the water. Since this food source is basically free and WWTPs are located around the country, a WWTP makes an ideal factory for growing beneficial bacteria for inline treatment of CSO, improvements to their own treatment process, or for the digestion of biosolids produced in the wastewater treatment process according to the invention.

The beneficial microorganisms grown at a WWTP can also be used for other environmental purposes that include but are not limited to disaster response to storm events such as hurricanes.

In addition to the adverse effects on WWTP operations, storm events cause major problems with microorganism contamination, mold production, toxic organic contamination, and mosquitoes. Beneficial microorganisms grown at a WWTP can be used to address all of these problems. For example, the efficiency of Bacillus Thuringiensis Israelensis (BTI) and Bacillus Sphaericus have been reported in a paper by Lacey L A, J. Am. Mosquito Control Association, 2007; 23 (2 Suppl); 133-63 to be effective in a variety of habitats against multiple species of mosquitoes. Also, the EPA documents that BTI is used across the United States for mosquito control and is approved for aerial spraying. BTI has been shown to be effective in reducing mosquito larval population and could be effective in controlling mosquitoes carrying Zika, Dengue, and Chikungumya viruses. The most effective way to use BTI to control mosquitoes is to grow BTI at a WWTP in large quantities to be applied globally.

Beneficial microorganisms are best grown in conditions with a readily available food supply that does not contain competing microorganisms like pathogenic bacteria found in municipal solid wastes. Therefore, it is preferred to disinfect the solid waste supplied to the WWTP to convert the solid waste into a clean food supply, so that desirable microorganisms can grow without competition from undesirable microorganisms.

While there may be other methods for disinfecting and making a food supply ideal for growing microorganisms, the preferred embodiment of the present invention is to use hydrodynamic cavitation for these purposes. Hydrodynamic cavitation involves agitating water with high energy to form bubbles. When the bubbles subsequently collapse, extremely high temperature and pressure conditions are created, which destroy undesirable microorganisms which would otherwise compete with the select beneficial microorganisms, which are introduced after the hydrodynamic cavitation step. Hydrodynamic cavitation also will lyse cells to destroy the cell wall and release liquid protoplasm, which is an ideal liquid food for growing microorganisms.

The effectiveness of a biological treatment system is a function of the type of microorganisms and the conditions that foster the growth of a healthy population of microorganisms. The two basis types of microorganisms are mobile organisms that are free floating and fixed microorganisms that become attached to some solid surface. The effectiveness of a biological treatment system is improved when there is a combination of both mobile and fixed microorganisms. Increasing the surface area for fixed microorganisms to attach to increases the concentration of microorganisms and therefore the effectiveness of the biological treatment process. Surface area is increased by the addition of biocarriers that are usually made of plastic with a high surface area and these can be either fixed or floating. To increase the ability of a conveyance system to act as a biological reactor requires the attachment of fixed biocarriers to the inside of the conveyance system so the microorganisms can remain in the conveyance system to accomplish biological treatment.

Work performed by Evoqua with their Captivator system (Patent Application No. US 201210043277A1) using a contact stabilization tank followed by a Dissolved Air Flotation system, and Veolia with their BioActiflo system (Enhanced Biological Treatment of Storm Flows, Filtration +Separation, March/April 2010), using a contact stabilization tank followed by Actiflo, both showed that MLSS from a WWTP can adsorb DC and remove TSS to meet regulatory requirements for BOD (biological oxygen demand) and TSS (total suspended solids) removal. Their goal was to treat Combined Sewer Overflow (CSO) from storm events that causes the discharge of raw sewage into U.S. waterways. In summary, Evoqua accomplished this task using their Captivator system to treat CSO by using a contact stabilization tank that contained aerated MLSS followed by DAF (dissolved air flotation) to remove TSS. Veolia accomplished this same task by also using their BioActiflo system to treat CSO by using contact stabilization tank that contained aerated MLSS followed by their Actiflo system to remove TSS. Because the Captivator system is in-line with the WWTP AST process, it reduces the biological load on the WWTP. The BioActiflo system is parallel to the WWTP AST process and does not reduce the biological load on the WWPT AST process. The process presented herein uses adsorbents and probiotic within the conveyance system and therefore reduces the biological load on the WWTP AST process.

The present invention improves on both the Captivator (Evoqua) and BioActiflo (Veolia) technologies because by using the wastewater conveyance system as a vessel where MHC, WTR, probiotics, or MLSS can adsorb and biologically treat DC in-line before the CSO reaches the WWTP, the need for a contact tank is eliminated. In addition, under normal conditions, using the wastewater conveyance system for pretreating wastewater before it reaches the WWTP will reduce the biological load on the WWTP and will reduce wastewater conveyance system problems like odor, corrosion, and buildup of FOG. None of these benefits are provided by competing Captivator or BioActiflo technologies.

As mentioned, one of the main shortcomings of AST is that the secondary clarifier causes limitations on the hydraulic capacity of AST because MLSS does not settle well. An enhancement to AST developed by Evoqua is the Biomag system (“Bioreactor Booster”, Casey Whittier, Water & Wastes Digest, Mar. 11, 2020) that adds magnetite to the aeration basin so the magnetite becomes embedded into the MLSS, forming a magnetic floc, and therefore increases its settling rate in the secondary clarifier. The settled magnetic floc is removed and the flocculating polymer bonds are mechanically broken to separate the magnetite from the MLSS. The magnetite is returned to the aeration basin for reuse and the MLSS is either returned to support the biological process as Returned Activated Sludge (RAS) or disposed of as Waste Activated Sludge (WAS). An improvement on the Biomag process according to the present invention is to again separate the magnetite for reuse but to take the separated MLSS and either deliver it upstream, preferably via a pipeline installed inside the wastewater conveyance system, because MLSS has been proven to adsorb DC from sewage, as described above or processing the MLSS by HTC to produce HC to also adsorb DC. Another alternative is to eliminate the process of separating magnetite from MLSS and process the magnetic floc with HTC to produce MHC and use this either in the aeration basin or use it upstream in the wastewater conveyance system.

According to an important aspect of the present invention, as mentioned above, magnetic floc from MHRC that contains magnetite and either MLSS from a WWTP or organic solids from the clarification of sewage using MHRC is an ideal material for producing MHC using HTC. However, according to another aspect of the present invention, magnetic floc is also ideal for the production of specialty bricks. An Australian research team from RMIT University in Melbourne Australia (Mohajerani, “Recycling Biosolids to Make Sustainable Bricks”, Society and Environment, 22 Jan. 2019), has demonstrated that clay-fired bricks containing biosolids could offer a sustainable solution for both brick-manufacturing and wastewater treatment industries. This research showed that manufacturing bricks with biosolids uses only half the amount of energy it takes to make conventional clay-fired bricks. Bricks made from biosolids thus not only cost less to produce, they have a lower thermal conductivity than all-clay bricks and therefore transfer less heat, which could potentially improve the environmental performance of buildings.

The volumes of biosolids produced globally each year is vast; more than 9 million tons is produced by the European Union each year, 7.1 million tons of biosolids is produced by the US annually, while Australia produces 327,000 tons a year. According to this study, there is a significant opportunity to recycle these biosolids into a beneficial product, namely bricks.

Research by S. O. Adeosun, et. al. in a paper titled “Refractory Behaviors of Magnetite-Kaolin Bricks” The Minerals, Metals & Materials Society, 2016 investigated the use of magnetite in the production of bricks. The conclusion of this research showed that magnetite, kaolin, and plastic clay are suitable for the production of insulating firebricks. Other applications for bricks that contain magnetite are for thermal storage and protection from gamma radiation. Using magnetic floc as an additive in the production of bricks will provide organics as well as magnetite to enhance the value of the bricks produced.

Research by Mohammed O. Ramadan, et. al. in a paper titled “Reuse of Water Treatment Plant Sludge in Brick Manufacturing” Journal of Applied Sciences Research 4 (10): 1223-1229, 9 (2008) concluded that by operating at the temperatures commonly practiced in the brick kiln, 50 percent by weight was the optimum addition of Water Treatment Residuals (WTR), to produce bricks superior to those available in the Egyptian market.

Therefore, it has been proven that materials such as biosolids and magnetite from different sources are beneficial for brick production. However, according to the invention, a novel source of both organics and magnetite is magnetic floc from MHRC that contains both organics and magnetite, which are beneficial in the production of bricks. Not only does this produce a valuable brick product, but it also provides a reuse option for organic wastes and reduces the amount of clay consumed in brick production.

Research by Tomi Turner, et. al. in a paper titled “Potential Alternative Reuse Pathways for Water Treatment Residuals: Remaining Barriers and Questions—A Review” Water Air Soil Pollution: 230-270, 2019 reported that the ever increasing need for clean water is resulting in vast quantities of Water Treatment Residuals (WTR), mainly containing aluminum or iron salts that neutralize the charge of solids contained in water so they can coagulate and settle more rapidly in a gravity clarifier, that are being disposed in landfills after only one use. This patent application proposes three additional uses for WTR after it has been used to clarify drinking water, as follows: (1) use of iron coagulants to clean sewage, (2) addition of WTR into a wastewater conveyance system to adsorb heavy metals, hydrogen sulfide, nutrients, and organics, and (3) use of the exhausted WTR recovered by clarification using MHRC in the production of bricks.

WTR are mostly produced by the use of inorganic coagulants in the treatment of drinking water to remove TSS, color, pathogens, odor, and colloidal particles. Each year millions of tons of WTR are produced and there has been significant efforts to find reuses for this waste. In a paper written by J. A. Ippolito et al. titled “Drinking Water Treatment Residuals: A Review of Recent Uses”, Journal of Environmental Quality, 40: 1-12, January 2011, it was reported that WTR is effective in removing phosphorus from wastewater, as a soil additive, and in removal of heavy metals and arsenic from wastewater. However, there is no suggestion in the public domain of the use of WTR to treat wastewater in its conveyance system and then removed and used to enhance brick production.

The use of magnetite to clean sewage is not new. Priestley et. al. U.S. Pat. No. 4,981,593, describes the Sirofloc system that mixes hydroxylated magnetite (i.e., having negative hydroxide ions on the surface layer of the magnetite after being treated with a caustic material) with sewage. The organic material in the sewage was treated with acid to form a positive charge on the particle surfaces that was adsorbed on the negatively charged hydroxylated magnetite particles during mixing, and a clarified liquid was formed and separated from the solids. The Priestley patent goes on to teach that the organic material may be separated from the magnetic particles before or after treatment in an anaerobic digestion system. FIG. 4 in the Priestley patent shows a treatment system, which includes a detailed description of the magnetite regeneration process. In summary, the Sirofloc process system as described in the Priestley patent involves adding acid to wastewater to impart a positive charge on the solid particles contained therein. Magnetite in the Sirofloc process was hydroxylated by treatment with a caustic to clean the magnetite and to impart a negative charge on its surface. The negatively charged magnetite attracts the positively charged particles, which clarifies the wastewater. This system is complicated and requires the use of acid to cause the particles in wastewater to have a positive charge and caustic is used to clean the magnetite and give it a negative surface charge. Laboratory tests conducted by the inventor proved that acidified magnetite effectively clarified sewage with the use of a polyelectrolyte flocculating polymer. This is a novel divergence from the Priestley patent, and is simpler and more cost effective. In laboratory tests, the initial TSS for the sewage was 216 ppm and following use of acidified magnetite and a cationic polyacrylamide flocculating polymer, the clarity (TSS) of the treated sewage was 8 ppm. Another advantage over the Priestley patent is this patent does not need the use of an inorganic coagulant as required by claim 6 of the Priestley patent that describes the use of an additional inorganic coagulant (i.e. iron or aluminum) to provide multivalent cations that are mixed with the sewage.

### Objects and Summary of the Invention

Aside from the preferred embodiment or embodiments disclosed below, this invention is capable of other embodiments and of being practiced or being carried out in various ways. Thus, it is to be understood that this invention is not limited in its application to the details of construction and the arrangements of components set forth in the following description and illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

It is therefore an object of this invention to provide a novel and efficient method to replace AST with MHRC and HTC to treat wastewater.

It is therefore an objective of this invention to provide a novel and efficient method to biologically treat wastewater.

Furthermore, it is an objective of this invention to use bioaugmentation (the use of microorganisms to help in the biological treatment of pollution), which includes bacteria and phages (viruses) in the treatment of wastewater.

Furthermore, it is an objective of this invention that bioaugmentation includes a mixture of ideally selected microorganisms to biologically treat wastewater, preferably a group of facultative bacillus bacteria that are film-forming.

Furthermore, it is an objective of this invention to use bioaugmentation to treat wastewater in its conveyance system, which may include pipelines, canals, and streambeds, in order to provide sufficient residence time for effective biological treatment.

Furthermore, it is an objective of this invention to add bioaugmentation to the conveyance system as close to the source of pollution as possible but preferably at the source.

Furthermore, it is an objective of this invention to grow the mixture of select microorganisms near to a source of organic material as food for the microorganisms and near to the source of pollution.

Furthermore, it is an objective of this invention to use biosolids produced at a WWTP to grow select microorganisms for use within the WWTP and to deliver the microorganisms into the wastewater conveyance system via a pipeline contained within the conveyance system.

Furthermore, it is an objective of this invention to disinfect the biosolids from a WWTP and convert them into a food source to grow selected beneficial microorganisms used to biologically treat wastewater.

Furthermore, it is an objective of this invention to add one or more flow control devices to increase the holding capacity of the wastewater conveyance system, which will allow more time for the mixture of select microorganisms to perform their water treatment functions.

Furthermore, it is an objective of this invention for the flow control device to also level out the flow rate of CSO and storm water from the conveyance system and other retention facilities so this water can be slowly clarified and disinfected if necessary over an extended period of time.

Furthermore, it is an objective of this invention to affix biocarriers to the inner surfaces of the wastewater conveyance system or to place the biocarriers anywhere within the conveyance system to enhance the growth of beneficial biofilm.

Furthermore, it is an objective of this invention to provide a screening system to remove large and floatable solids from wastewater prior to clarification and disinfection.

Furthermore, it is an objective of this invention to use high-rate clarification and flocculating polymers, preferably in a system that uses magnetite as a ballast material in a high-rate clarification system, to remove fine suspended solids contained in wastewater so that treated wastewater can be directly discharged into the environment without the need of a WWTP.

Furthermore, it is an objective of this invention for the wastewater first entering the conveyance system at the beginning of a storm event, which typically is the most contaminated and accordingly termed “first flush”, to be diverted into the WWTP for immediate treatment.

Furthermore, it is an objective of this invention to irrigate land with water and suspended solids coming from the high-rate clarification system.

Furthermore, it is an objective of this invention to provide additional storage and treatment capacity in structures located in a stream or riverbed to supplement the treatment capabilities of the existing conveyance system.

Furthermore, it is an objective of this invention to use biosolids, especially the microorganisms collected from the high-rate clarification system, to enhance biological treatment capacity of a WWTP.

Furthermore, it is an object of this invention to use WWTPs as a means of producing select microorganisms to be used to treat bacteria contaminated water and mosquito larvae resulting from hurricanes or other extreme storm events.

Furthermore, it is an object of this invention to use the “process water” from HTC to grow a mixture of microorganisms (probiotics) used to remove DC from wastewater, especially in wastewater conveyance systems.

Furthermore, it is an object of this invention to use MHC, MLSS, MHC, HC, WTR and probiotics separately or in combination to reduce odor, corrosion, and FOG in wastewater conveyance systems.

Furthermore, it is an object of this invention to use wastewater conveyance systems as pretreatment systems prior to MHRC or prior to an existing WWTP using AST, preferably by installing a pipeline inside the wastewater conveyance system, to deliver MHC, MLSS, WTR, HC, or probiotics upstream to allow sufficient time for treatment prior to final treatment in a centralized WWTP.

Furthermore, it is an object of this invention to use HTC to convert solid organic wastes such as but not limited to septage, animal waste, MLSS, food waste, and grease trap waste into HC and, with the addition of magnetite originating from MHRC, to produce MHC.

Furthermore, it is an object of this invention to combine MHRC for clarification to remove TSS with HTC to produce MHC to remove nutrients, CEC, pathogens, and DC contained in wastewater, all in order to serve as a replacement for AST.

Furthermore, it is an object of this invention to leave as much nutrients (phosphorus, potassium, nitrogen) as possible in the treated wastewater so it can be used effectively for irrigation.

Furthermore, it is an object of this invention to combine the use of organics contained in sewage with iron based materials (WTR and magnetite) used in the treatment of wastewater to produce bricks.

Normally, HTC is used to process wet organic wastes to produce HC, a concentrated carbon product with reduced water content. There are multiple uses for HC including: as a low cost solid fuel, as a soil amendment, or as an adsorbent to clean water. Of these, the most valued use of HC is as an adsorbent to clean water. However, a preferred form of HC is when it is made magnetic to facilitate its recovery from wastewater. Producing MHC that contains carbonized carbon and a magnetic material can be accomplished in many ways, such as adding naturally mined magnetite or adding a nano-sized magnetite produced from ferric and ferrous salts. However, the most cost effective and novel way to produce MHC is to use magnetic floc from MHRC that contains organic TSS, magnetite, polymer, and spent MHC that has adsorbed DC or other contaminants contained in colloidal particles. This provides the necessary magnetic material (magnetite) and carbon material necessary for HTC to use heat and pressure to produce a carbonized magnetic adsorbent such as MHC.

The ratio of carbon to magnetite can be easily adjusted by adding other sources of organic wastes like food wastes, MLSS, animal waste, grease trap wastes, or septage. The adsorptive properties of the MHC can also be affected by the type of solid organic waste added, the operating temperatures and pressures in the HTC, and the addition of other chemicals to affect the surface chemistry and porosity of MHC.

Certain distinct aspects and elements of the system represented by this invention are known individually in the art, such as: (1) the use of in-line bioaugmentation using probiotics to reduce the biological load on a VWVTP, (2) the use of magnetite to enhance clarification by increasing solids settling rate, (3) use of HTC to produce HC from wet organic wastes, (4) production of MHC by the addition of iron salts or magnetite to HTC, (5) using probiotics for in-line treatment in wastewater conveyance systems to reduce odor, corrosion, and buildup of FOG, (6) the properties of MLSS to adsorb DC, (7) the adsorption properties of WTR, and (8) the adsorptive properties of MHC. However, these known elements have neither been combined nor contemplated in the public domain as proposed according to the present invention, specifically by (1) processing magnetic floc (solid organics, magnetite and flocculating polymers) from MHRC in a HTC to produce MHC to remove DC from wastewater, (2) adding MHC, MLSS, WTR, HC, and probiotics either separately or in combination, preferably via a pipeline installed inside a wastewater conveyance system, to adsorb and biologically treat DC before the wastewater stream reaches a final clarification step using MHRC, (3) replacing AST and Chemically Enhanced Primary Treatment (CEPT—discussed further below), with MHRC and HTC to treat municipal wastewater, (4) using “process water” from HTC to grow probiotics used in-line in a wastewater conveyance system for biological pretreatment, (5) using organics contained in wastewater, specifically municipal wastewater, and combining with iron products, specifically magnetite for clarification, WTR, and MHC that are used to treat the wastewater and adding these products into brick production, and (6) using MHRC and HDC and its associated “process water” to produce probiotics that are used in water conveyance systems to treat Combined Sewer Overflow (CSO) and Separate Sewer Overflow (SSO) streams instead of using solids contact technology followed by DAF or Actiflo high rate clarification technologies.

According to the present invention, wastewater that contains TSS and DC is treated with a novel process that replaces traditional AST. The process involves circular economics where solid contaminants contained in wastewater are clarified with MHRC and the resulting magnetic floc, containing magnetite, polymer, and suspended solids, is processed with HTC to produce MHC for water treatment. See FIG. 1, discussed in detail below.

More specifically, MHC is introduced upstream into a wastewater conveyance system with sufficient time and agitation due to in-line flow turbulence for DC to be adsorbed onto the MHC. The main advantage of adsorption over biological treatment is reduced treatment time and the adsorption of CEC. Biological treatment takes hours and adsorption usually takes minutes. The speed and capacity to adsorb DC is dependent on the surface area of the adsorbent, the physical properties of that surface such as pore size, the amount of agitation, and the chemical nature of its surface. Most importantly, the greater the adsorbent's surface area, the greater the speed and capacity of treatment. Therefore, smaller adsorbent particles are better but small particle adsorbents are more difficult to remove from water. This is the reason that MHC is better than HC because the particle size can be made extremely small by mechanical grinding or by HDC and still be removed easily from water magnetically. The needed residence time for MHC to adsorb DC is far less than the amount of time takes for wastewater to flow through its conveyance system. Agitation can be controlled by a mixer located in an adsorber tank such as in the Captivator and BioActiflo technologies or through water flow turbulence within the conveyance pipeline as proposed in this patent application.

Wastewater that contains TSS, MHC, MLSS, WTR, and probiotics either separately or combined flows through a conveyance system to a MHRC that uses polymer to flocculate all the solid particles that have adsorbed most or all of the DC. The slurry of solids (magnetic floc) that is removed by the MHRC is then processed by HTC to produce MHC.

HTC is operated at a temperature and pressure that converts the TSS, waste organics, and DC adsorbed onto the incoming MHC into new MHC and “process liquids” that contain nutrients such as nitrogen, phosphorus, and potassium, all vital for plant and bacteria growth.

The newly produced MHC is then used to adsorb DC contained in wastewater flowing through its conveyance system for a minimum of two minutes to assure adsorbence of DC onto the MHC.

A major advantage of using MHC in place of AST is that MHC can be modified to target specific contaminants such as CEC, which include toxic organics, pesticides, hormones, pharmaceuticals, nutrients, or heavy metals, while the bacteria used in AST are more singularly purposed and are best at removing BOD, phosphorus, and nitrogen and not CEC or other non-biodegradable DC.

While MHC is effective in adsorbing DC and will improve operating conditions in a wastewater conveyance system by reducing odor, adsorbing hydrogen sulfide that causes corrosion, and adsorbing FOG so it does not build-up on wastewater conveyance walls, biological treatment using probiotics grown on magnetic floc that has been treated with HDC and includes WTR and MLSS's also has the ability to treat wastewater in its conveyance system and therefore is also an effective element of this invention.

HTC “process liquids” separated from solid MHC are high in nutrients such as nitrogen and phosphorus and can grow probiotics used to treat DC contained in wastewater. Biological treatment using probiotics can be done in-line much the same way MHC, WTR, and MLSS are used to remove DC from wastewater and can also reduce odor, corrosion, and FOG in the wastewater conveyance system.

As mentioned above, according to the invention the conventional AST system shown in FIG. 7a is replaced by the simplified system shown in FIG. 7b, using hydrodynamic cavitation (HDC) to process magnetic floc into a food source to grow probiotics. For comparison purposes, a typical AST system as shown in FIG. 7a is complex and costly and includes primary and secondary clarification steps, aerobic biological treatment, and disinfection. HDC processing will disinfect the magnetic floc so pathogens contained therein will be destroyed and will not compete with the growth of probiotics and also reduce the particle size of the magnetite to increase its surface area to enhance its speed and capacity to adsorb DC.

In most cases, environmental regulations require secondary biological treatment of sewage and CSO/SSO to achieve an 85% reduction of Biological Oxygen Demand (BOD—a measure of the amount of organic pollutants present). This is one reason that CEPT has not been readily accepted to treat municipal wastewater or CSO/SSO because CEPT is not a biological process and is only used in situations where waterway eutrophication is not considered a problematic issue, such as ocean discharge. More specifically, CEPT uses chemical coagulants such as aluminum or iron salts that contain a positive electric charge to neutralize the negative charge of particles in wastewater and therefore allows the particles to be more easily flocculated. Therefore, while AST is usually necessary to comply with the biological treatment requirement to achieve regulatory compliance, AST is not the lowest-cost alternative for the treatment of sewage or CSO/SSO and is not a feasible treatment option for developing countries that need nutrients in irrigation water to grow food. In-line treatment with MHC, WTR, MLSS and probiotics according to this invention will satisfy this biological treatment requirement and is the lowest cost treatment option.

As mentioned above, the preferred mixture of microorganisms (probiotics) to be used in the practice of this invention are non-pathogenic, and non-toxic, which includes but is not limited to B. Subtiles, B. Licheniformes, B. Indicus, B. Coagulans, B. Cereus, and B. Clausii. These bacteria are commonly found in nature, especially in soil. They are facultative by nature, meaning that they do not need oxygen to survive and be productive. Therefore, they are effective in both aerobic and anaerobic environments and reduce odor and corrosion in the conveyance systems by consuming chemicals, especially sulfur, contained in the wastewater.

As also mentioned above, phages are viruses that infect and kill microorganisms. Sulfur-reducing bacteria (SRB) cause significant harm in a wastewater conveyance system because they convert sulfates contained in the water into sulfides that react to form hydrogen sulfide, a major cause of odor, and sulfuric acid, a major cause of corrosion. Phages infect and control the growth of SRB but can be selected to do so while not harming beneficial microorganisms.

Bioaugmentation performed in the conveyance system is effectively accomplished by growing microorganisms onsite using “process liquids” from HTC that have been disinfected by high temperature in the HTC or by using Hydrodynamic Cavitation (HDC) to process magnetic floc from MHRC or MLSS from AST. HDC uses the explosive force of collapsing microbubbles to disinfect and lyse organic cells to release liquid nutrients vital to the growth of probiotics and in agriculture.

Once wastewater has been treated in its conveyance system to remove DC by the use of probiotics, WTR, MHC, and MLSS, clarification is necessary to meet total suspended solids (TSS) limits (usually 30 ppm) and Biological Oxygen Demand (BOD) limits (usually 30 ppm) associated with solid particles and colloids. Since a large percentage of TSS and BOD are in the form of fine solids that do not settle well, the most effective method according to this patent for removing these fine suspended solids and colloidal particles is MHRC, using magnetite and flocculating polymer to form a magnetic floc that can then be separated from the water using magnetic techniques such as permanent magnets.

As also mentioned above, because of the sheer magnitude of the problem and the cost of adequate solutions, regulations regarding wastewater are constantly changing. It appears, according to present EPA regulations, that primary clarification and disinfection are acceptable solutions for water treatment if bacteria levels can be reduced to sufficiently low levels, and if the BOD of the wastewater can be biologically reduced by at least 85% to under 30 ppm. This can be accomplished by using MHC, WTR, MLSS, and/or probiotics separately or in combination to perform adsorption and biological treatment processes in the wastewater conveyance system, followed by MHRC for particle clarification as described in this patent application. Probiotics can also be added to the treated effluent to substitute for chemical disinfection and improve the biological health of the receiving waterway.

CSO is a major environmental problem because in some locations, as in older cities in the U.S. Northeast, storm water and sanitary wastewater comingle in one pipeline, so that during a storm event the receiving WWTP cannot treat the increased volume of water. Therefore, raw sewage can bypass the WWTP and be discharged directly into a waterway. Various methods to alleviate this problem have been proposed, such as the construction of containment structures, which have not been completely satisfactory. Clarification and disinfection, while helpful, have not been accepted by the EPA, which has consistently required some level of biological treatment to achieve an 85% BOD reduction.

A solution to meet the 85% removal rate requirement that includes biological treatment, is referred to in the art as MLSS contact stabilization technology. One such technology bypasses CSO around the WWTP into a solids contact tank that contains aerated MLSS that is reported to meet the biological treatment requirement, followed by high rate clarification using the sand ballast clarification technology (Actiflo—Veolia BioActiflo) to remove TSS. Another such technology also meets the biological treatment requirement by placing in series with the WWTP a solids contact tank followed by Dissolved Air Flotation (DAF—Evoqua Captivator) to reduce the biological and solids load on the WWTP, but not the hydraulic load. However, these solutions are expensive since they require the construction of a large solids contact tank that needs a constant inventory of aerated MLSS to be maintained during periods of non-use (dry weather). In addition, these treatment processes do nothing to treat the wastewater conveyance system and the BioActiflo system does not reduce the biological load on the WWTP during normal operation. Using sand ballast technology produces a dilute waste (0.1-0.3 wt. % solids) that places an additional hydraulic load on the WWTP. The equipment required for DAF clarification is 5-10 times the size of a comparable MHRC and uses 5-10 times more energy. Another major problem with sand ballast clarification and DAF is the time required for startup (10-15 minutes). A better solution to treat CSO and SSO and as a replacement for Captivator and BioActiflo technologies is provided by the present invention. This invention uses MHC, WTR, MLSS and probiotics separately or in combination to convert the existing wastewater conveyance system into an in-line adsorber and bioreactor, followed by MHRC for high rate clarification. In summary, this application describes the lowest cost option that: (1) reduces the biological treatment load on the VW TP during normal operation, (2) reduces problems with odor, corrosion, and FOG in the wastewater conveyance system, (3) meets TSS and BOD discharge limits, and (4) complies with the required 85% reduction of BOD using biological treatment methods. MHRC is superior to sand ballast technology because it produces a waste that is ten times more concentrated, requires equipment that is one half the size for the same flowrate, starts up quicker, and does not require the use of a chemical coagulant. MHRC is an improvement on DAF technology because it has a smaller footprint, starts up quicker, uses less energy, is mechanically less complicated; does not require the use of a chemical coagulant, and produces better water quality. The combination of treatments using MHC and WTR for adsorption, and MLSS and probiotics for biological treatment, all applied separately or in combination within the wastewater conveyance system followed by MHRC for clarification is a better alternative to existing technologies to treat CSO/SSO and as a replacement for CEPT, Captivator, BioActiflo, and AST.

While there may be other methods for disinfecting sludge and making a food supply ideal for growing microorganisms, the preferred embodiment of the present invention is to use either HTC “process liquids” or HDC-treated magnetic floc from MHRC, or HDC treated MLSS for growing microorganisms. HTC involves using heat and pressure to disinfect pathogens while HDC uses collapsing microbubbles to disinfect pathogens and lyse organic cells.

Over 20 million tons of biosolids from the treatment of sewage are produced each year and a large percentage of these biosolids are disposed in landfills that can otherwise be combined with magnetite and processed with HTC to produce MHC. Magnetic floc sourced from MHRC to clarify organic wastewater is a prime source of materials to produce MHC using HTC.

Millions of tons of WTR are produced each year by using iron coagulants mostly to clean drinking water. According to an important aspect of the present invention, these sources of iron-based materials (magnetite and WTR) are used to clean wastewater; after this use, the excess of carbon and iron products can be used in the production of bricks. The annual production of 1500 billion bricks globally requires over 3.13 billion cubic meters of clay soil—equivalent to over 1000 soccer fields dug 440 meters deep. According to this invention, a practical solution is provided for the utilization of the world's excess of organic waste organics and iron wastes in the production of bricks. The advantage of this use is that carbon wastes produced from cleaning water are reused, which reduces disposal in landfills, reduces energy usage to produce bricks, reduces the consumption of clay, and provides a valuable reuse of WTR.

Specifically, according to the invention, MHC, WTR, MLSS, and probiotics are injected separately or in combination into a wastewater conveyance system to adsorb DC, specifically organics and heavy metals. The wastewater is then clarified using MHRC to produce magnetic floc to remove TSS and adsorbed DC. The magnetic floc is then either used as a source of iron and carbon for the production of bricks or reconstituted along with other sources of waste carbon by processing in HTC to produce MHC that is reinjected back into the wastewater conveyance system to remove DC.

Hydrodynamic Cavitation (HDC) is the process of vaporization, bubble generation, and bubble implosion, which occurs in a flowing liquid as a result of a decrease and subsequent increase in local pressure. HDC will only occur if the local pressure declines to some point below the saturated vapor pressure of the liquid and subsequent recovery above the vapor pressure.

HDC can be performed by different processes such as but not limited to passing a liquid through a constricted channel at a specific flow velocity or by mechanical rotation of an object through a liquid such as in a pump. The process of bubble generation, and the subsequent growth and collapse of the cavitation bubbles results in very high temperatures and pressures on the surface of the bubbles for a very short time. The overall liquid medium environment, therefore, remains at ambient conditions. HDC can be used to enhance chemical reactions or propagate certain unexpected reactions because hydroxyl radicals are generated in the process due to disassociation of vapors trapped in the cavitating bubbles. According to an important aspect of the present invention, HDC is used to disinfect waste by high pressure, temperature, and the presence of hydroxyl radicals so that the organic waste can then be used to grow useful probiotics or in other agriculture applications. In addition, under such extreme conditions, the cells of organic substances rupture and release liquid protoplasm that contains nutrients beneficial for the growth of probiotics and food.

In practice of the invention, DC are adsorbed on solid adsorbents, specifically WTR, MHC, and MLSS either separately or in combination in either the wastewater conveyance system or as an alternative in an agitated tank and are removed from water using MHRC, producing a magnetic floc. This magnetic floc contains magnetite, flocculating polymer, and TSS that has adsorbed DC and are removed from sewage using MHRC.

The normal process for recovering magnetite from magnetic floc produced by MHRC employs mechanical agitation, which breaks the polymer bonds and separates the magnetite from waste TSS. Clean magnetite is then returned to the MHRC for reuse and the separated non-magnetic solids are normally disposed of.

According to the invention, a new and novel process is provided wherein the magnetic floc is treated in an improved way to make it more suitable for recovery and reuse. Magnetic floc is processed using HDC to disinfect the waste so that pathogens do not compete with the growth of beneficial probiotics. HDC also lyses organic cells to liberate liquid nutrients that can be used by the probiotics as a food source.

Once the magnetic floc is processed with HDC, the resulting magnetite is separated by a magnetic collection device and is reused in the wastewater conveyance system to adsorb DC and to resupply the MHRC with fresh magnetite. HDC will also reduce the particle size and therefore increase the surface area of the magnetite to make it a more effective adsorbent. The non-magnetic solid wastes that have been disinfected and lysed are dewatered. The filtrate from dewatering organic wastes that have been treated by HDC is rich in liquid nutrients, useful to grow probiotics. The separate solids are disinfected and heavy metals can be easily removed to make the solids suitable as an organic fertilizer.

There are many suitable ways to dewater the HDC-treated solid organics. The two main ways are mechanical dewatering that consumes electricity and flocculating polymer, and passive dewatering that uses solar energy for drying. The lowest cost dewatering method is a reed bed planted with vetiver, often called a “miracle grass”, which requires minimum amounts of electricity for pumping and requires no flocculating polymer. Planting the reed bed with vetiver provides other benefits such as a source of animal feed, supply of suitable liquid nutrients and compost to increase food production, reduces soil erosion, provides an alternative solid fuel, and treats wastewater. Probiotics grown from reed bed filtrate are then used to treat wastewater in the wastewater conveyance system and excess filtrate still contains valuable nutrients that are good for irrigation in food production.

MHRC is a key component in treating wastewater as proposed in this application because it enhances the flocculation process and speeds the rate by which solids can be removed from water. TSS is attached to magnetite with the use of a flocculating polymer, preferably a polyacrylamide, but for this process to be effective two things have to occur. First there has to be sufficient agitation to overcome repulsive forces that prevent particles from flocculating together. The amount of agitation applied has to be sufficient to keep the magnetite in suspension in a mix tank or conveyance system. Second, the repulsive forces that prevent particles from flocculating together have to be minimized.

The main repulsive force that is effected by the addition of magnetite is surface charge. Opposite charges attract and similar charges repel. Since the surface charge of fine particles and organics is mainly negative, the common practice is to add either a ferric or aluminum ion, both of which have a plus three charge; it is this positive charge that negates the negative surface of particles and colloids in sewage. However, the use of iron or aluminum coagulants increases the amount of sludge produced and makes it more difficult to reuse the sludge for beneficial purposes. Controlling the surface charge by acidic treatment is one way to place a positive surface charge on magnetite to replace the use of chemical coagulants.

Magnetite is amphoteric, meaning that it can act as either acid or base. The surface charge of magnetite is normally measured by its zeta potential. When the net charge of a particle's surface is zero, this is called the Point of Zero Charge (PZC). For magnetite this PZC is between 6-7 pH. When the pH is above the PZC, the surface charge of magnetite is negative; when the pH is below the PZC, the surface charge of magnetite is positive. Therefore, when magnetite is acidified and the pH is below about 6, the surface charge is positive and is effective in attracting the negatively charged particles found in sewage.

### BRIEF DESCRIPTION OF THE DRAWINGS

Other objects, features and advantages will occur to those skilled in the art from the following description of a preferred embodiment and with reference to the accompanying drawings, in which:

FIG. 1 shows an overview of the treatment technologies employed in a novel way according to the invention and how MHRC, HTC, WTR, biological agents, and MHC are integrated either separately or individually into one system that economically treats wastewater.

FIG. 2 shows the details of a Biological Grow System that uses “process liquids” from HTC and organic waste solids to produce biological agents herein also referred to as probiotics.

FIG. 3 shows a first embodiment of a Magnetic High Rate Clarifier (MHRC) for removing magnetic floc, which is composed of TSS, magnetite, and flocculating polymer to remove suspended solids from water using permanent magnets.

FIG. 4 shows a second embodiment of a Magnetic High Rate Clarifier (MHRC) for removing magnetic floc, which is composed of TSS, magnetite, and flocculating polymer to remove suspended solids from water using gravity.

FIG. 5 shows a system for using Water Treatment Residues (WTR), MHC, magnetic floc from MHRC, and probiotics to treat wastewater in a conveyance system and to improve the operating conditions in the wastewater conveyance system, followed by clarification using MHRC to produce a magnetic floc that is used in brick production and as a feedstock to HTC to produce MHC.

FIG. 6 shows an alternative process to treat wastewater using MHRC for clarification and Hydrodynamic Cavitation (HDC) to treat the magnetic floc produced from MHRC and to dewater the magnetic floc with a reed bed to produce solid organic fertilizer and nutrient rich liquids, which may be used to grow probiotics in turn used to treat wastewater in the wastewater conveyance system.

FIG. 7a shows a typical state of the art WWTP using AST that includes clarification, biological treatment, and disinfection.

FIG. 7b shows a comparison system to AST according to this invention that uses MHRC for clarification and HDC to process magnetic floc from MHRC to grow probiotics and reduce the particle size of magnetite so they can be used in-line in the conveyance system to treat sewage.

FIG. 8 shows an overview of the treatment technologies employed according to the invention and how they are integrated to produce the desired result of economically treating wastewater.

FIG. 9, comprising FIGS. 9(a) and 9(b), shows a flow control device that can be adjusted to change the pool level in the conveyance system, which will vary the holding capacity of the conveyance system.

FIG. 10 shows the extension of a wastewater conveyance system and its placement inside a riverbed.

FIG. 11 shows a delivery system that takes select microorganisms grown at a WWTP and transports the select microorganisms either through a pipeline placed within the wastewater conveyance system upstream of the WWTP or into the effluent of the WWTP.

### DETAILED DESCRIPTION OF THE INVENTION

While this invention is susceptible to implementation in many different forms, there is shown in the drawings and will herein be described in detail specific embodiments, with the understanding that the present disclosure of such embodiments is to be considered as an example of the principles of the invention, and is not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

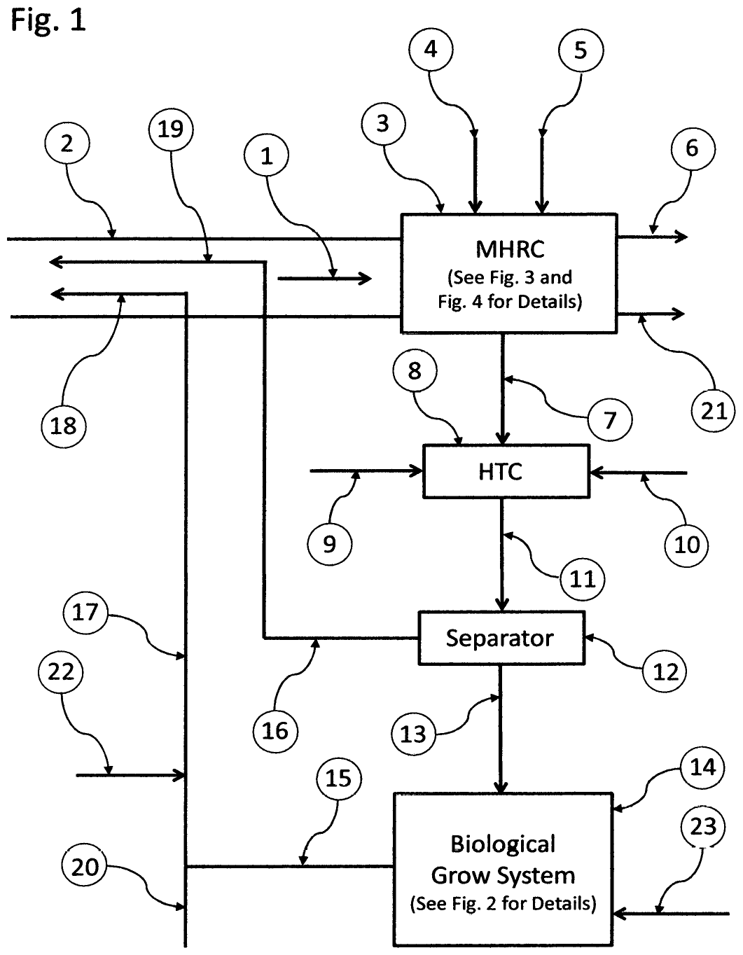

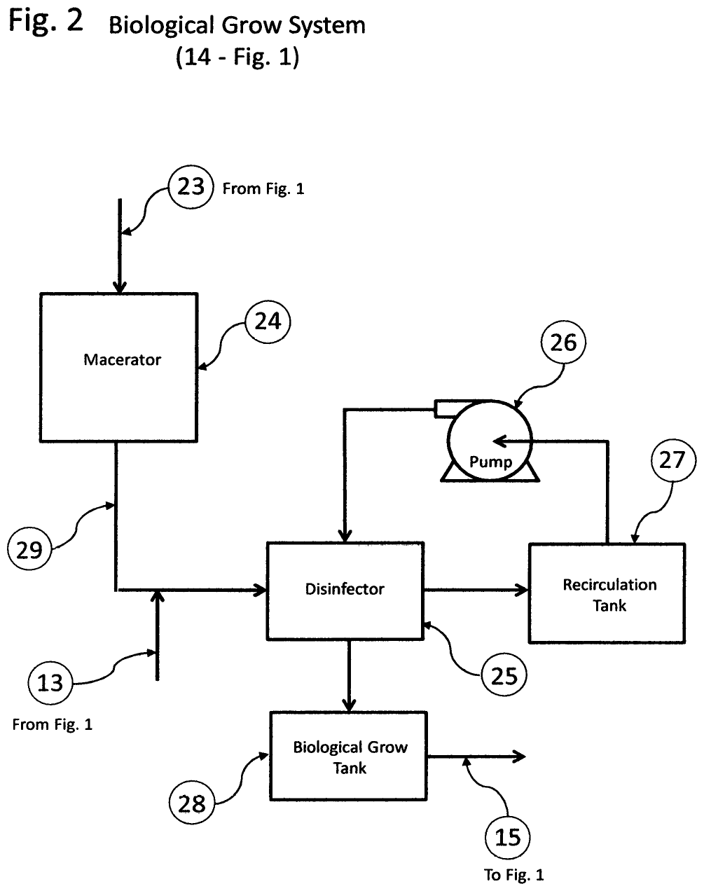

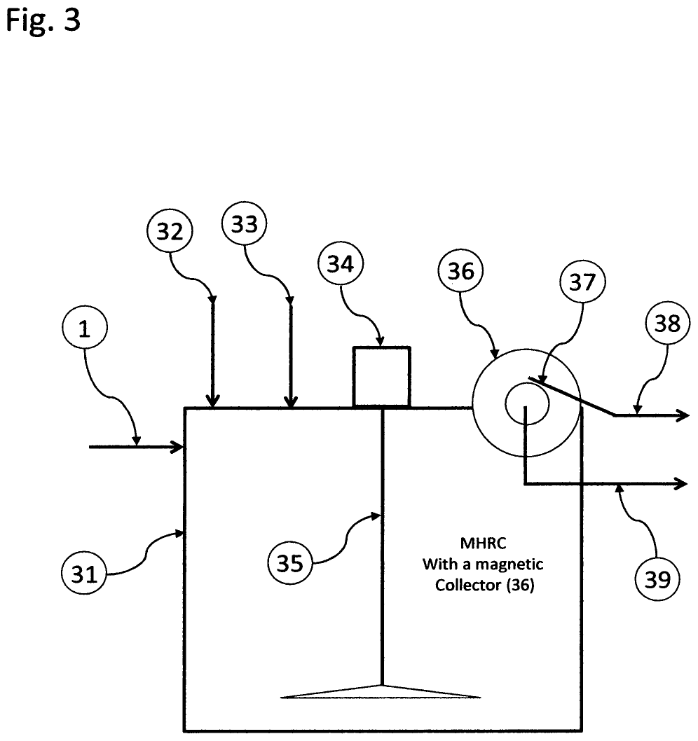

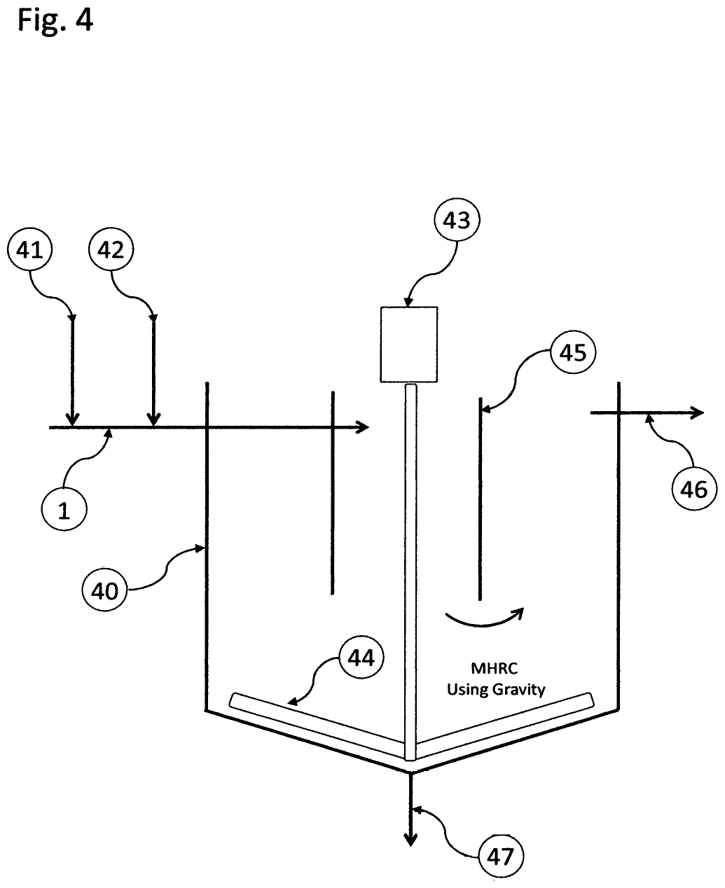

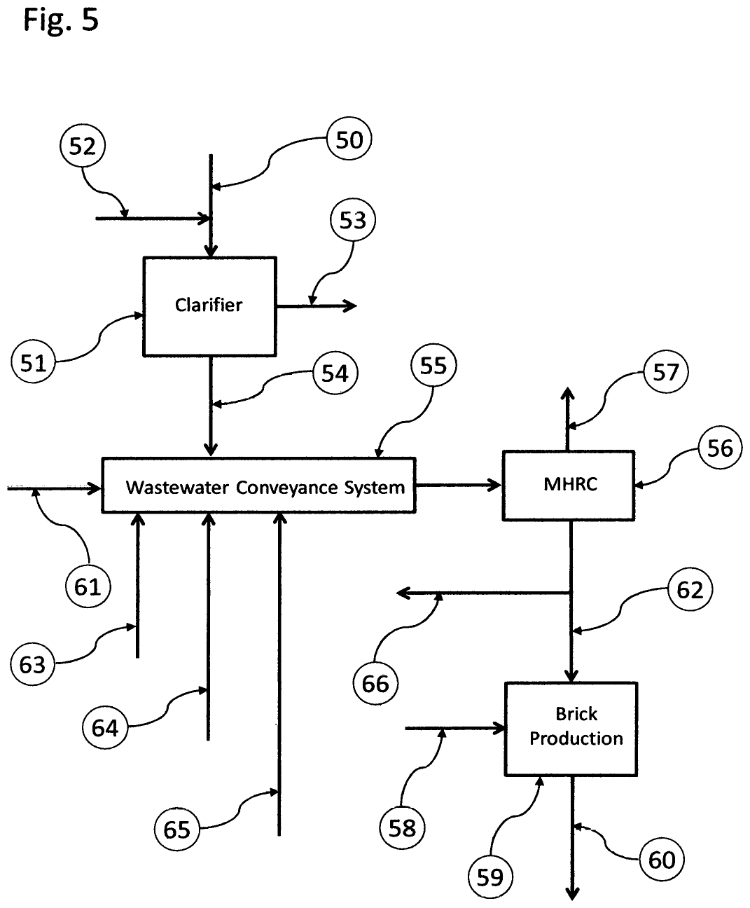

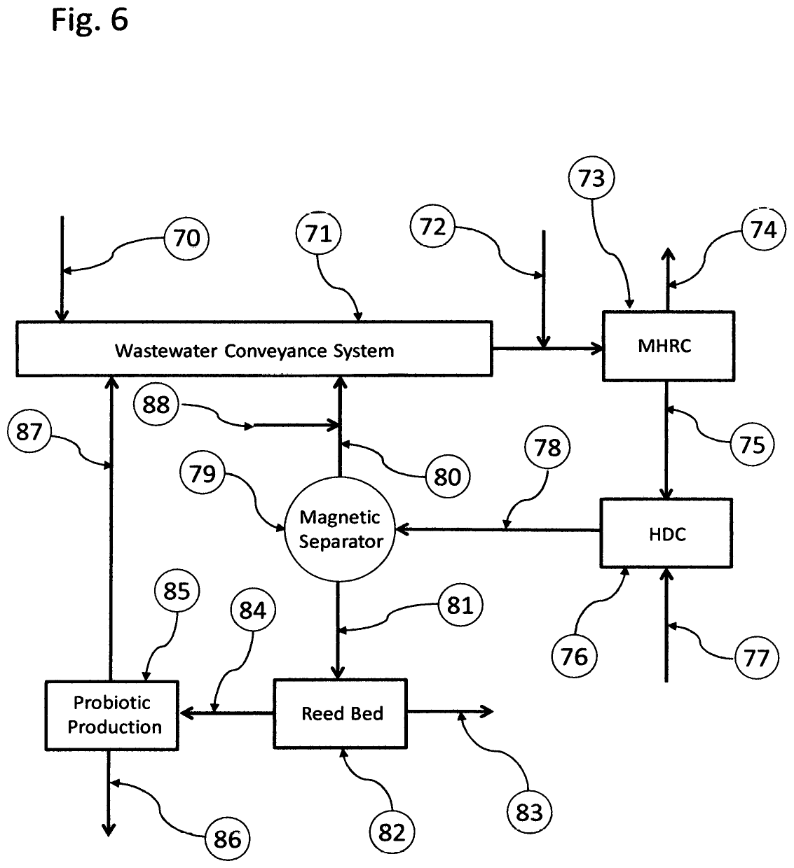

FIG. 1 shows a wastewater treatment system according to the invention, wherein a stream of wastewater (1) that has been treated in-line by the addition either combined or separately of MHC (16), MLSS (20), WTR (22), and probiotics (15) flows into a Magnetic High Rate Clarifier (MHRC) (3) (different embodiments of which are detailed in FIGS. 3 and 4) where treated water that contains TSS is combined with a flocculating polymer (4) and magnetite (5) to form a magnetic floc (7) composed of magnetite (5), polymer (4) and TSS including organic solids, WTR, MHC, and biological agents such as probiotics. The floc is separated from the cleaned water in the MHRC (3) as described in detail below with respect to FIGS. 3 and 4. The clarified water (6) is discharged and the separated magnetic floc (7) flows to a Hydrothermal Carbonizer (HTC) (8). Magnetic floc (7) discharged (21) from the MHRC can also be used in the production of bricks as shown in FIG. 5 or be supplied to a HDC as shown in FIG. 6. The HTC (8) performs a thermochemical process used to convert wet biomass such as sewage biosolids into a solid product called HydroChar (HC). The HTC (8) employs pressurized water at relatively low temperatures between 180 degrees centigrade and 250 degrees centigrade and at or below saturated pressure. The HTC process mainly entails decarboxylation, dehydration, and polymerization. Removing carboxyl and hydroxyl groups reduces the oxygen to carbon ratio, which makes the HC more carbon-dense and therefore more suitable as a solid fuel or as an adsorbent. The HTC (8) also produces “process liquids” that are high in nutrients, especially phosphorus, that can be recovered. If the HC produced by HTC (8) contains magnetite, the product is termed Magnetic Hydrochar (MHC). Other sources of organic wastes (9) such as food waste, grease trap waste, WWTP biosolids, septage or animal waste can also be processed in the HTC (8) to increase the carbon to magnetite ratio of the MHC. The preferred ratio of carbon to magnetic material in MHC is 2:1. Special additives such as iron or other catalysts (10) can be added to the HTC (8) to improve the adsorption properties of MHC. MHC and “process liquids” (11) flow from the HTC (8) into a separator (12) that separates MHC (16), preferably by gravity, from the “process liquids” (13). A slurry of MHC (16) is pumped into a pipeline (19) that is preferably placed inside the wastewater conveyance system (2). The “process liquids” (13) flow from the Separator (12) into a Biological Grow System (14), detailed in FIG. 2, that grows probiotics and other microorganisms with the nutrients contained in the “process liquids” (13). Biological agents (15) containing probiotics and other microorganisms grown in the Biological Grow System (14) may combine with MLSS (20) from a WWTP or WTR (22) in a pipeline (17) that connects to a pipeline (18) that is also placed inside the wastewater conveyance system (2). The purpose of this combined flow of biological agents (15), MLSS (20), and WTR (22) either separately or combined is to adsorb and biologically treat DC flowing (1) in the wastewater conveyance system (2), which essentially converts the wastewater conveyance system into a wastewater pretreatment system.

FIG. 2 shows the details of the Biological Grow System (14) shown in FIG. 1 for growing biological agents (15), comprised primarily of bacteria and phages, to biologically treat wastewater inside a wastewater conveyance system. Organic waste (23), specifically, solid organic wastes (i.e. food waste, WWTP biosolids, septage, animal waste, or grease trap waste), are mixed in a Macerator (24) designed to blend and make homogeneous the organic wastes (23). This homogeneous product (29) combines with “process liquids” (13) from a HTC (8) and flows to a Disinfector (25), more specifically a Hydrodynamic Cavitation (HDC) system that uses collapsing microbubbles to disinfect organic wastes (29) so that pathogens contained therein do not compete with the growth of the microorganisms in the Biological Grow Tank (28). The organic wastes (23) and HTC “process liquids” (13) that have been treated in the Disinfector (25) then flow into a Recirculation Tank (27) and are returned using a Pump (26) back to the Disinfector (25) to allow the organic wastes to be lysed and disinfected multiple times. Once the organic cells have been suitably disinfected to remove pathogens and have been lysed to release additional liquid nutrients, the treated organic wastes flow to a Biological Grow Tank (28) to feed a mixture of microorganisms, preferably facultative bacillus bacteria and phages as discussed above. These microorganisms (15), then flow through a pipeline (17-FIG. 1) into the wastewater conveyance system (2-FIG. 1).

FIG. 3 shows the details of a first embodiment of a Magnetic High Rate Clarifier (MHRC) that uses permanent magnets as a final filter to prevent the discharge of magnetic floc, and to allow recovery of the magnetite for reuse. Wastewater (1) that that has been pretreated in-line to adsorb and biologically remove DC flows into a MHRC tank (31). Polymer (32) and magnetite (33) are added to the MHRC tank (31) and agitated with a motor (34) and mixer blade (35) to cause a magnetic floc (38) to form. The magnetic floc (38) is collected on a plurality of rotating magnetic disks (36) and as the disks rotate, a scraper (37) mechanically removes the magnetic floc (38) from the magnetic disks (36) and the magnetic floc (38) is discharged. Clear water (39) flows from the center of the magnetic disks (36) and is discharged (39).

FIG. 4 shows another embodiment of a MHRC that uses gravity to separate the magnetic floc. Wastewater (1) that has been pretreated in-line to adsorb and biologically remove DC and can contain MHC, WTR, MLSS, organic TSS, and probiotics are combined in-line with flocculating polymer (41) and magnetite (42) to form magnetic floc. The magnetic floc flows into a MHRC tank (40) where the magnetic floc settles by gravity and the settled magnetic floc is moved by a motor (43) powered scraper (44) to the center of the MHRC tank (40) and discharged (47). A baffle (45) causes the magnetic floc to settle to the bottom of the MHRC (40) tank and clarified water is discharged (46). Some water flows out the bottom of the tank in the form of a slurry but its flow rate is limited because it is transferred with a pump that limits the flow.

FIG. 5 shows the process for reusing WTR that contains TSS and spent coagulants that usually contain iron or aluminum to manufacture bricks. Water (50) is combined with an iron or aluminum coagulant (52) that neutralizes the negative charge of particles contained in the water (50) that then flows into a Clarifier (or MHRC) that discharges clarified water (53) and WTR (54). The WTR (54) flows into a Wastewater Conveyance System (55) where it combines with MHC (63), MLSS (64) produced by AST, and probiotics (65) grown using “process liquids” from HTC, separately or in combination. This combination of solids in the Wastewater Conveyance System (55) then flows to a MHRC (56) where the magnetic floc (62) produced as discussed above goes into Brick Production (59) and can undergo additional treatment to produce specialty adsorbents and catalysts (66). Clay (58) is added and bricks (60) are produced. In this case, the WTR replaces some of the water needed in brick-making, reduces clay consumption, provides carbon fuel, and supplies metal additives to enhance the quality of bricks.

FIG. 6 shows a further embodiment of a water treatment system according to the invention that combines technology with biology. Wastewater (70) flows through a Wastewater Conveyance System (71) where it comes into contact with probiotics (87) and magnetite (80) and then is contacted with flocculating polymer (72) to produce a magnetic floc that flows into a MHRC (73). Clarified water (74) exits MHRC (73) and magnetic floc (75) flows to a HDC (76) for the purpose of disinfecting pathogens contained in the magnetic floc, lysing organic cells to promote the growth of probiotics, breaking polymer floc bonds, and reducing the particle size of magnetite to improve its function as an adsorbent, all as described above. Another feature of this process is that organic wastes (77) such as food waste, grease trap waste, MLSS, animal waste, and septage can be processed in the HDC (76) to convert these wastes into solid organic fertilizer (83) and the filtrate (84) that contains valuable nutrients is used to grow probiotics. The magnetic floc (78) that has been processed by HDC (76) flows to a Magnetic Separator (79) that is composed of permanent magnets designed to recover magnetite (80) that is reused in the Wastewater Conveyance System (71). Prior to reuse of the magnetite (80), acid (88) is added to give a positive charge to the surface of the magnetite so it is effective in attracting the negatively-charged particles contained in sewage. Non-magnetic solids (81) from the Magnetic Separator (79) flow to a Reed Bed (82) that dewaters the solids (81) using solar energy and gravity. The dewatered solids (83) are useful as an organic fertilizer and the filtrate (84) provides nutrients to grow probiotics in a Probiotic Production (85) system. Produced probiotics are useful to irrigate (86) crops and useful to treat wastewater (87) in the Wastewater Conveyance System (71).

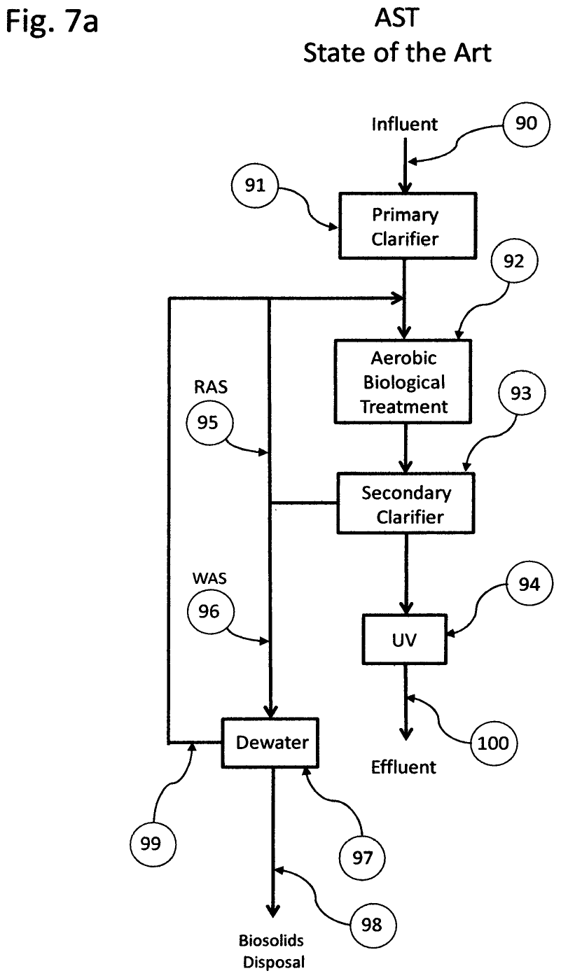

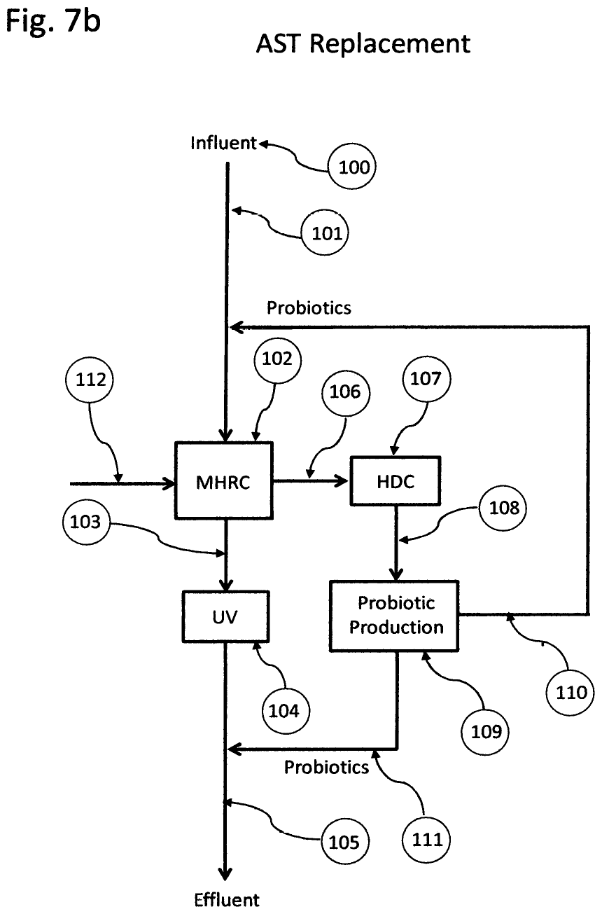

FIG. 7a shows a state of the art WWTP employing AST. Wastewater (90) flows into a primary clarifier (91) and solid waste (not shown for simplicity) is either discharged as WAS (96) or used in an anaerobic digester (not shown) to produce methane for electricity generation. Clarified water from the Primary Clarifier (91) is then biologically treated in an Aerobic Biological Treatment System (92) where bacteria convert DC into biosolids called Mixed Liquor Suspended Solids (MLSS). MLSS then flows to a Secondary Clarifier (93) where solids are separated and used as Returned Activated Solids (RAS) (95) to enhance the biological treatment process or Waste Activated Solids (WAS) (96) that is disposed. Clarified water from the Secondary Clarifier (93) is then disinfected by UV (94) or other disinfection method and discharged (100). Primary and secondary solids are dewatered (97) with the filtrate (99) returned to the head of the AST for additional treatment and the Biosolids (98) are normally disposed of.

FIG. 7b shows for comparison purposes an AST replacement system according to the invention, which reduces: (1) capital and operating costs (2) sludge production, (3) greenhouse gas emissions, (4) physical footprint, and (5) operating complexity and is especially suitable for applications in developing countries. Wastewater (101) flows through a conveyance system (107) and into a MHRC (102) where clarified water flows to UV (104) for disinfection and separated solids flow to a HDC (103) where they are disinfected and lysed. Flow from the HDC (103) goes to a Probiotic Production system (105) that grows probiotics according to this invention. Probiotics and magnetite are pumped upstream into the Influent Conveyance System (107) and probiotics only are pumped into the effluent line (106).

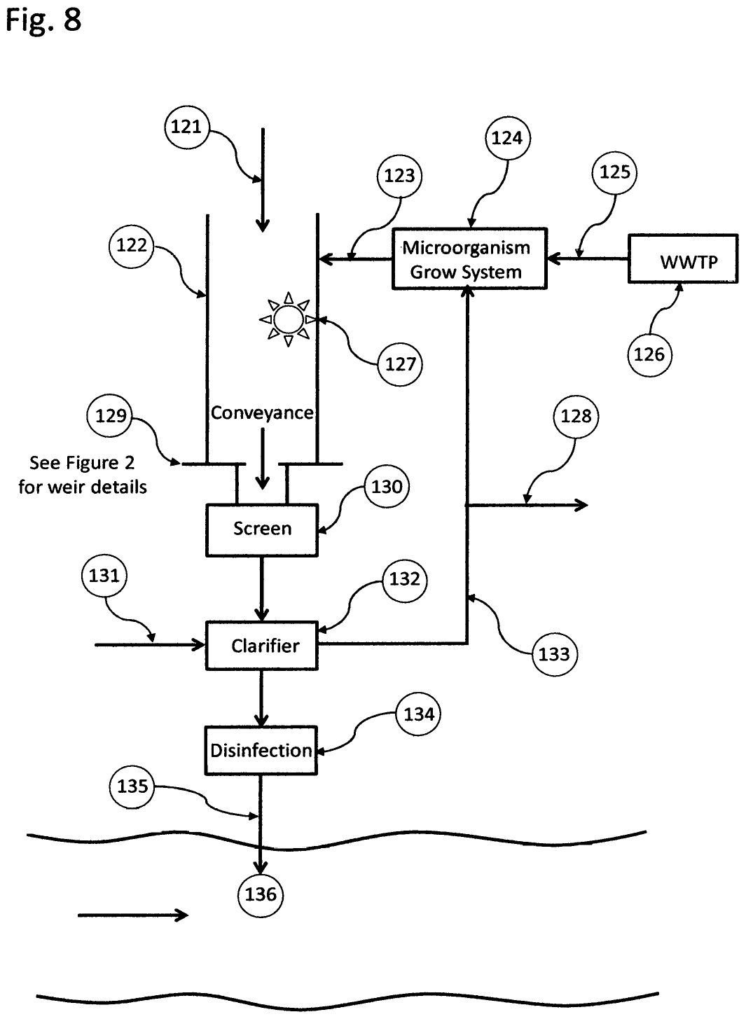

FIG. 8 shows wastewater (121) flowing through a water conveyance system (122), such as a lake, river, impoundment structure or the like, into which is injected a mixture of microorganisms (123) that treat the wastewater inside the water conveyance system (122) to consume undesirable organics. As indicated above, these microorganisms may include facultative bacilli and phages. The treated wastewater then flows through a flow control device (129) that controls the flow rate and the level of wastewater in the water conveyance system (122) to give microorganisms time to work. Biologically treated wastewater then flows through a screen device (130) that removes large solids and floatables. The screened wastewater then flows into a high-rate clarification device (132) where polymer (131) is added to flocculate fine suspended solids for removal. Flocculants may be employed together with magnetite to remove the flocculated solids, e.g., as described in applicant's co-pending application Ser. No. 14/612,635, filed Feb. 3, 2015, and incorporated herein by this reference. The clarified wastewater then flows through a disinfection device (134) and then through a conveyance system (135) and into a river (136). Solids removed by the high-rate clarification device flow (132) and as an alternative biosolids (125) from a WWI? (126) flow to a microorganism grow system (124). Also shown in this FIG. 8 are biocarriers (127) attached to the inside surface of the conveyance system (122) to increase the surface area for the growth of biofilm.

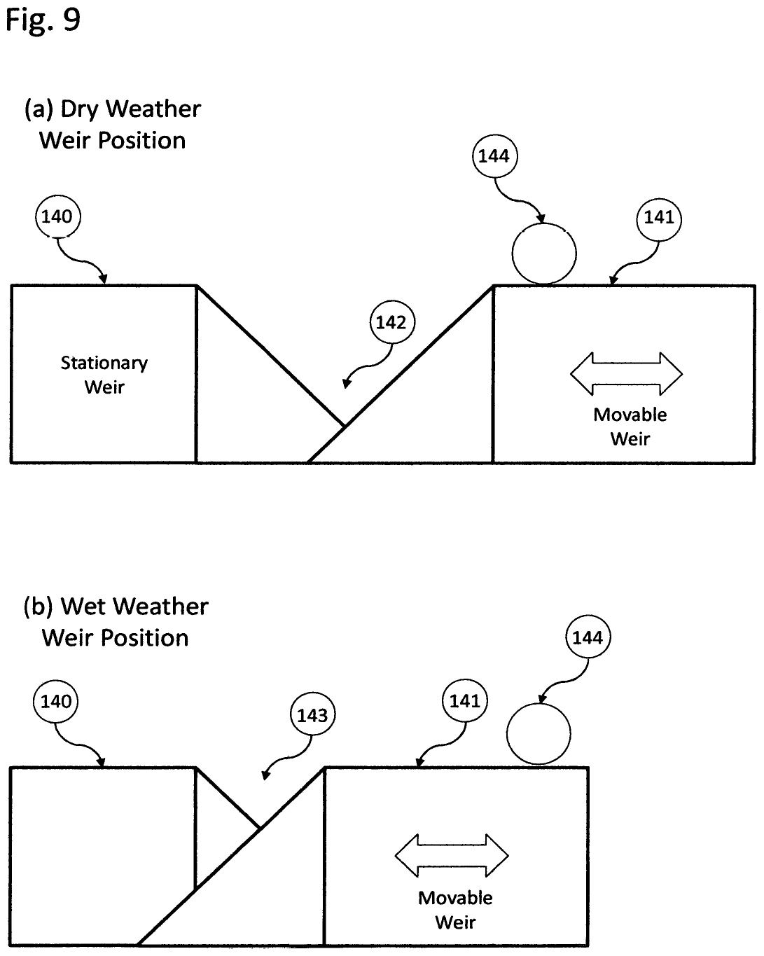

FIG. 9, comprising FIGS. 9(a) and (b), shows a flow control device that can be adjusted to change the water level in the conveyance system. The main purpose of the flow control device is to back up wastewater into the conveyance system to allow for additional time for biological treatment and to control and level out the flow of wastewater for subsequent treatment. The preferred flow control device is a V notch weir composed of two parts. One part (140) is a stationary weir and the other part is a movable weir (141) sealed to the stationary weir to prevent most water leakages. The flow control device has basically two positions. One position (FIG. 9(a)) determines a low-level pool height (142) that is employed during dry weather and another position (FIG. 9(b)) determines a high-level pool height (143) that is employed during wet weather events. This allows the residence time of water in the conveyance system to be controlled to be substantially constant despite varying flow rates in dry and stormy weather. A mechanical device (144) powers the movable weir (141).

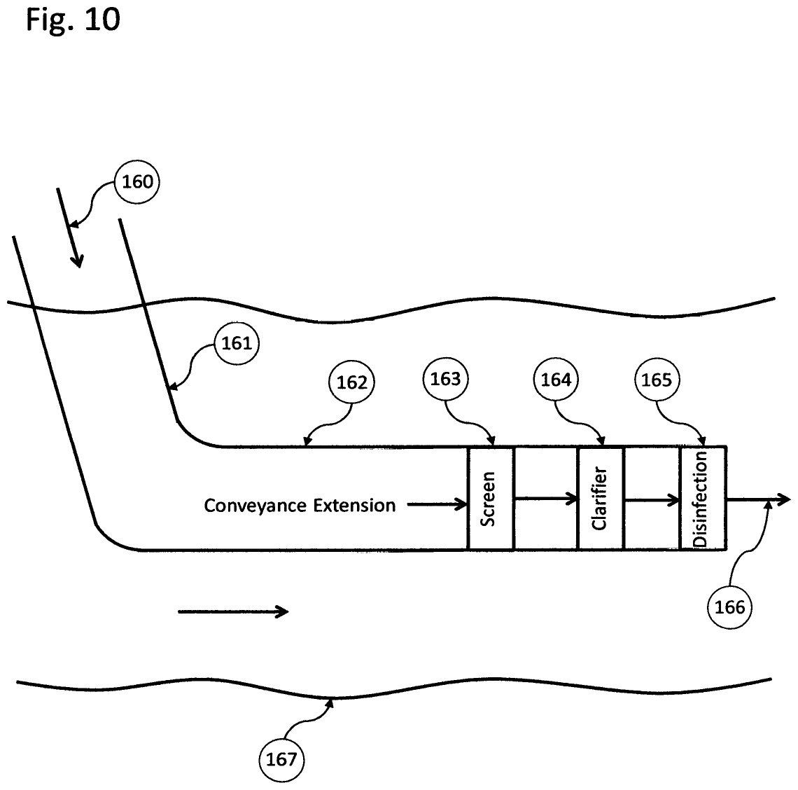

FIG. 10 shows biologically treated wastewater (160) flowing through a conveyance system (161) and into a conveyance system extension (162) that has been positioned within or alongside a riverbed and where biological treatment is taking place. The biologically treated wastewater within the conveyance system extension (162) is then screened (163) to remove floatables, then clarified (164) to remove suspended solids, and then disinfected (165) to kill pathogens before the treated water is discharged (166) into a riverbed (167).

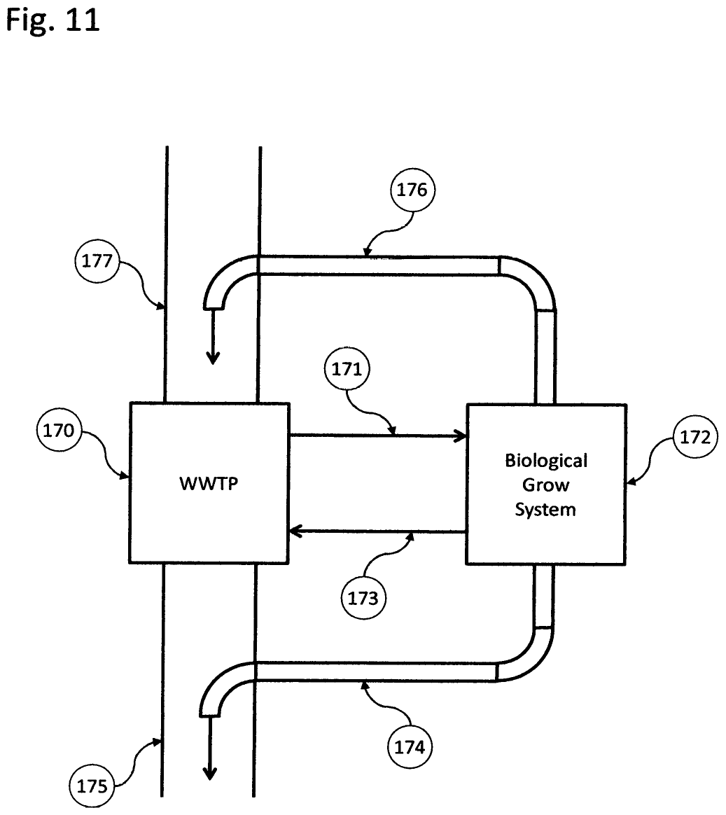

FIG. 11 shows a biological grow system (172) that receives organic wastes (171) from a WWTP (170) to produce select microorganisms that are either returned (173) to the WWTP (170) for use and/or piped (176) through a pipeline inserted inside the conveyance system (177) for inline bioaugmentation and/or piped (174) to the effluent of the WWTP (175).

While a preferred embodiment of the invention has been disclosed, the invention is not to be limited thereto.

What is claimed is:

1. A method for purifying water, comprising the steps of:
providing a select probiotic production system, for growing probiotics useful in treating organic contaminants in water, wherein said probiotics are grown using lysed and disinfected biosolids or other organic wastes as food for the probiotics;
providing a pipeline for supplying said probiotics to the stream of water at a given location;
employing a magnetic high rate clarifier (MHRC), downstream of said given location, for attaching magnetite particles to dissolved and suspended solids in the water using a flocculating polymer to form a magnetic floc, and for subsequently removing said magnetic floc from the water employing a magnetic attraction technique, leaving an effluent water stream;
providing a hydrothermal carbonization (HTC) system, wherein the removed magnetite particles and dissolved and suspended solids are processed to yield a magnetic hydrochar material that is high in carbon and is a useful adsorbent that is used to treat the stream of water in its conveyance system;
employing a disinfecting system wherein the effluent water stream from the MHRC is disinfected and discharged for use; and
lysing and disinfecting solid organic wastes, so as to serve as food for growing probiotics.

2. The method of claim 1, wherein the biosolids from a sewage treatment plant or other organic wastes employed to grow probiotics are disinfected and lysed by methods including mechanical, thermal, electrical, sonic methods, and hydrodynamic cavitation, to kill competing microorganisms, including pathogenic bacteria and sulfur reducing bacteria (SRB) and to increase the food value of the organic wastes by releasing the cell protoplasm contents beneficial for growing the probiotics.

3. The method of claim 1, wherein the organic wastes that have been disinfected and lysed to make suitable for growing probiotics includes biosolids from a sewage treatment plant, septage, grease trap wastes, and food wastes.

4. The method of claim 1, wherein an adjustable flow control device in said water conveyance system is provided to control the accumulation of water within the conveyance system, so as to control the residence time of water in the conveyance system,.

5. The method of claim 1, wherein the probiotics contain bacillus bacteria selected from the group consisting of B. Subtiles, B. Licheniformes, B. Indicus, B. Coagulans, B. Cereus, and B. Clausii.

8. The method of claim 1, wherein the conveyance system may be a pipeline, canal, or a natural watercourse that may be extended by a man-made canal or other conveyance system added alongside or within a river or stream to increase the residence time so biological treatment can continue to treat water before it enters a river or stream.

7. The method of claim 4, wherein the flow control device comprises an adjustable weir so as to create either a smaller reservoir of water during dry weather conditions or a larger reservoir of water during wet weather conditions, allowing control of the residence time of the water in the reservoir to provide time for the probiotics to treat the water to desired levels.

8. The method of claim 1, wherein process liquids from HTC are used to grow probiotics or as a source of organic fertilizer.

9. The method of claim 1, wherein magnetic floc from a MHRC is hydrothermally carbonized in a HTC to produce magnetic hydrochar.

10. The method of claim 1, comprising the further steps of screening floatables before said MHRC step and disinfection after said MHRC.

11. The method of claim 1, wherein the MHRC removes fine suspended solids including bacteria and other microorganisms by the use of an anionic or cationic flocculating polymer and/or coagulant.

12. The method of claim 1, wherein the MHRC has a surface overflow rate greater than 10 gallons per minute per square foot.

13. The method of claim 1, wherein solids removed by MHRC are diverted into one or more containment structures that are either positioned in a riverbed that increases the biological treatment time or pumped to a nearby waste water treatment plant (WWTP) for further clarification, biological, and dewatering treatment.

14. The method of claim 1, wherein probiotics that have been removed by a MHRC are routed to a probiotic grow system to provide a constant source of probiotics to enhance the performance of the WWTP, to treat water upstream in the conveyance system, and also downstream of the WWTP to improve river water quality.

15. The method of claim 1, wherein the surface charge on the magnetite used in the MHRC is modified using chemicals or controlling pH so the magnetite acts as a reusable chemical coagulant to neutralize the charge on TSS and thus eliminates the use of chemical coagulants.

16. The method in claim 1, wherein said adsorbents include magnetite, MLSS, carbon and chemical based adsorbents, and water treatment residuals produced from the treatment of drinking water.

17. The method in claim 1, wherein the adsorbents are either macrosized greater than 5 microns or microsized such as nano particles.

18. The method in claim 1, wherein the adsorbents have been functionalized by chemical, thermal, or mechanical means to modify the surface of the adsorbent to selectively adsorb dissolved contaminants contained in water.

19. The method in claim 1, wherein the magnetic floc from MHRC is used in the production of fired brick.

20. The method in claim 1, wherein the pipeline used to deliver the probiotics upstream into the water conveyance system is installed inside the conveyance system or above ground.
